SEQUENCE LISTING (2 sequences)

SEQ ID NO: 1 - protein, 114 aa, Homo sapiens
MNVLLGSVVIFATFVTLCNASCYFIPNEGVPGDSTRKCMDLKGNKHPINSEWQTDNCETCTCYETEISCCTLVSTPVGYDKDNCQRIFKKEDCKYIVVEKKDPKKTCSVSEWII

SEQ ID NO: 2 - protein, 94 aa, Homo sapiens
SCYFIPNEGVPGDSTRKCMDLKGNKHPINSEWQTDNCETCTCYETEISCCTLVSTPVGYDKDNCQRIFKKEDCKYIVVEKKDPKKTCSVSEWII

(19) United States
(12) Patent Application Publication
(10) Pub. No.: US 2012/0022793 A1
(43) Pub. Date: Jan. 26, 2012

(54) BIOMARKERS FOR THE DIAGNOSIS OF PROSTATE CANCER IN A NON-HYPERTENSIVE POPULATION

(75) Inventors: Douglas BARKER, Winnipeg (CA); Katrin Atnikov, Winnipeg (CA); Yilan Zhang, Winnipeg (CA); Stephen Frost, Gurnee, IL (US)

(73) Assignee: Miraculins, Inc., Winnipeg (CA)

(21) Appl. No.: 13/189,270

(22) Filed: Jul. 22, 2011

Related U.S. Application Data
(63) Continuation-in-part of application No. 13/144,662, filed on Oct. 5, 2011.
(60) Provisional application No. 61/371,374, filed on Aug. 6, 2010.
(60) Provisional application No. 61/151,308, filed on Feb. 10, 2009.
(60) Provisional application No. 61/145,671, filed on Jan. 19, 2009.

(51) Int. Cl. G06F 19/10; G01N 33/574

(52) U.S. Cl. 702/19; 435/7.94

(57) ABSTRACT

The present invention includes the use of biomolecules for differential diagnosis of prostate cancer and/or non-malignant disease of the prostate. The present invention also provides methods for detecting biomolecules within a biological sample. The invention further includes kits for differential diagnosis of prostate cancer and/or non-malignant disease of the prostate in a biological sample.

This application is a Continuation-in-Part Application of U.S. Ser. No. 61/371,374, filed 6 Aug. 2010, and of U.S. Ser. No. 13/144,662, filed 14 Jul. 2011, which is a National Stage entry under 35 U.S.C. §371 of PCT/CA2010/000078, filed 19 Jan. 2010, which claims the benefit of U.S. Ser. No. 61/151,308, filed 10 Feb. 2009, and which also claims the benefit of U.S. Ser. No. 61/145,671, filed 19 Jan. 2009 and which applications are incorporated herein by reference. A claim of priority to all, to the extent appropriate is made.

### FIELD OF THE INVENTION

The present invention relates to diagnosing prostate cancer. More particularly, the present invention includes a method for differential diagnosis of prostate cancer from a non-malignant disease of the prostate and/or from a healthy prostate.

### BACKGROUND

Prostate cancer is one of the most common cancers to afflict men in western countries. In North America the incidence rate for prostate cancer in males is an estimated 166.7 per 100,000 per year, which accounted for an estimated 33% of all newly reported cancers in men in 2005 (American Cancer Society, 2005). The Canadian Cancer Society indicates that one in 7 men will develop prostate cancer, mostly after age 70 (Canadian Cancer Society, 2005). In 2005, American Cancer Society and Canadian Cancer Society estimated the mortality rate for this disease to be 20% (American Cancer Society, 2005; Canadian Cancer Society, 2005).

The current standard screening method for prostate cancer is the Prostate Specific Antigen (PSA) test, which can take the form of total PSA measurements, free:total PSA ratios, and PSA velocities (change in PSA levels over time) (Egawa et al., 1997; Djavan et al., 1999). Typically, an individual has been characterized as having an elevated risk for prostate cancer with a PSA level above 4.0 ng/mL (Gann et al., 1995). This can be refined to account for a number of factors, such as PSA levels increasing naturally with age (Oesterling et al., 1994). Clinicians must rely on complementary diagnostic tools because PSA screening is an imperfect means of diagnosis, is not indicative of pathological stage (Beduschi and Oesterling, 1997; Erdem et al., 2002-2003), and has poor specificity. The result is healthy patients being subjected to unnecessary testing and an increased financial and emotional toll of prostate cancer diagnosis. The primary diagnostic tools used in addition to PSA testing are the digital-rectal exam (DRE) and prostate biopsy. DREs are performed routinely in conjunction with PSA tests and biopsies to improve the accuracy of diagnosis (Scattoni et al., 2003). Prostate biopsies are the means of ultimate confirmation of diagnosis, but have significant complication rates (Rodriguez and Terris, 1998). The U.S. Preventative Services Task Force does not recommend the PSA test for routine screening. Despite the known shortcomings of PSA testing and significant amounts of research, there has been little improvement in the state of the prostate disease diagnostics. Thus, there is an unmet need for more accurate prostate disease diagnostics, particularly prostate cancer.

Prostate Secretory Protein (PSP94), also known as beta-microseminoprotein or inhibin-like peptide, is a basic 94 amino acid protein with a molecular weight of 10,704 (Seidah et al.). PSP94 is generated from a 114 amino acid precursor encoded by a nucleotide sequence located on chromosome 10 (Dube et al, 1987). Purified PSP94 isolated from seminal fluid migrates between 13-16 kDa on a polyacrylimide gel (Dube et al, 1987), and the difference in molecular weight is not due to glycosylation but due to the basic nature of the protein (Seidah et al, 1984). PSP94 is found in high concentration in prostate epithelial cells (Brar et al, 1988) and has been examined in serum and urine for its potential to be a cancer biomarker. Results from Kaighn et al. (1987) demonstrated that PSP94 was not detectable in a PC-3 cell line from human prostatic carcinomas. Furthermore, PSP94 in urine was decreased in men with late stage tumors using 24 hr collection (Teni et al, 1988; Teni et al, 1989; Trembley et al, 1987).

Abnormal protein levels in serum are indicative of prostate cancer, wherein the irregular or erratic control of PSP94 secretion from the prostate is correlated with neoplasia (Wu et al., 1999). Several groups explored PSP94 for the potential of becoming a relevant biomarker for prostate cancer (Dubé et al., 1987; Tremblay et al., 1987; Abrahamsson et al., 1988; Teni et al., 1988; Abrahamsson et al., 1989; Teni et al., 1989; von der Kammer et al., 1990; Huang et al., 1993; Hyakutake et al., 1993; von der Kammer et al., 1993, Maeda et al., 1994; Tsurusaki et al., 1998; Sakai et al., 1999). However, these groups were never successful at producing a reliable diagnostic test for prostate cancer utilizing PSP94.

### SUMMARY OF THE INVENTION

Recently, we identified a peak at 10750 M/Z by mass spectrometry that decreased in the urine of patients with prostate cancer. The peak corresponded to PSP94. We then developed an immunoassay to measure PSP94 in urine to develop a commercial assay to help minimize the number of prostate biopsies for men with PSA values between 0.0 and 10.0 ng/mL with a negative DRE.

An aspect of the present invention relates to methods for differential diagnosis of prostate cancer or non-malignant disease of the prostate by detecting PSP94 and PSA within a test sample of a given subject, comparing results with samples from healthy subjects, subjects having precancerous prostatic lesion, subjects with non-malignant disease of the prostate, subjects with localized cancer of the prostate, subjects with metastasised cancer of the prostate, and/or subjects with an acute or a chronic inflammation of prostatic tissue, wherein comparison allows for differential diagnosis of a subject as healthy, having a precancerous prostatic lesion, having non-malignant disease of the prostate, having localized prostate cancer, having a metastasised prostate cancer or having an acute or chronic inflammation of prostatic tissue. In an embodiment, the subject does not have hypertension.

One aspect of the invention includes a method for diagnosing prostate cancer in a subject comprising detecting a quantity, presence, or absence of PSP94 and total PSA in a biological sample; and classifying said subject as having or not having prostate cancer, based on said quantity, presence or absence of PSP94 and total PSA. In one embodiment, the step of classifying said subject comprises comparing the quantity, presence, or absence of PSP94 and total PSA with a reference value indicative of a prostate cancer. In an embodiment, the subject does not have hypertension.

A further aspect of the invention includes a method for differential diagnosis of prostate cancer and non-malignant disease of the prostate in a subject, comprising detecting a quantity, presence or absence of PSP94 and total PSA in a biological sample and classifying said subject as having prostate cancer, non-malignant disease of the prostate, or as healthy, based on the quantity, presence or absence of PSP94 and total PSA in said biological sample. In one embodiment, the step of classifying said subject comprises comparing a quantity, presence, or absence of PSP94 and total PSA with a reference value indicative of prostate cancer and a reference value indicative of a non-malignant disease of the prostate. In an embodiment, the subject does not have hypertension.

A further aspect of the invention includes a method for differential diagnosis of healthy, non-malignant disease of the prostate, precancerous prostatic lesion, localized cancer of the prostate, metastasised cancer of the prostate, and acute or chronic inflammation of prostatic tissue in a subject, comprising detecting a quantity, presence or absence of PSP94 and total PSA in a biological sample and classifying said subject as having non-malignant disease of the prostate, precancerous prostate lesion, localized cancer of the prostate, metastasised cancer of the prostate, and/or acute or chronic inflammation of prostatic tissue, or as healthy, based on the quantity, presence or absence of PSP94 and total PSA in said biological sample. In one embodiment, a step of classifying said subject comprises comparing a quantity, presence or absence of PSP94 and total PSA with a reference value indicative of healthy, non-malignant disease of the prostate, precancerous prostate lesion, localized cancer of the prostate, metastasised cancer of the prostate, acute inflammation of prostatic tissue or chronic inflammation of prostatic tissue. In an embodiment, the subject does not have hypertension. In a further aspect diagnosis includes differential diagnosis.

An aspect of the present invention relates to methods for evaluating a prognosis of prostate cancer in a subject. The methods comprise detecting a quantity of PSP94 and total PSA in a test sample; and classifying the progression of cancer. The present method permits differentiation of prostate cancer subjects with a good prognosis (high probability of recovery, becoming disease free) from subjects with a bad prognosis (low probability of recovery, cancer reoccurrence, metastasis). In an embodiment, the subject does not have hypertension.

In a further embodiment of the methods of the invention, a quantity, presence, or absence of PSP94 and total PSA are detected or quantified in a biological sample obtained from the subject utilizing an antibody to said biomarker.

In a further embodiment of the methods of the invention, a quantity, presence, or absence of PSP94 and total PSA are detected or quantified in a biological sample obtained from the subject through the use of an ELISA assay.

In a further embodiment of the methods of the invention, a quantity, presence, or absence of PSP94 and total PSA are detected or quantified in a biological sample obtained from the subject through the use of a BioPlex® Immunoassay (Bio-Rad Laboratories, Hercules, Calif.).

In a further embodiment of the methods of the invention, a quantity, presence, or absence of PSP94 and total PSA are detected or quantified through a use of a biochip.

In a further embodiment of the invention, a quantity, presence, or absence of PSP94 and total PSA are detected or quantified in an automated system.

In a further embodiment of the invention, a subject is a mammal. The subject may be a human.

In a further embodiment of the invention, a test or biological sample used according to the invention may be blood, blood serum, blood plasma, urine, semen, seminal fluid, seminal plasma, prostatic fluid, pre-ejaculatory fluid (Cowper's fluid), excreta, tears, saliva, sweat, bile, biopsy, ascites, cerebrospinal fluid, lymph, or tissue extract origin. In a further embodiment of the methods of the invention, the test and/or biological samples are urine, semen, seminal fluid, seminal plasma, prostatic fluid, pre-ejaculatory fluid (Cowper's fluid) samples, and are isolated from subjects of mammalian origin, preferably of human origin. In a still further embodiment of the invention, the test and/or biological samples are blood, blood serum, plasma and/or urine.

In a further embodiment of the invention the biological sample can be urine.

In a further embodiment of the present invention the urine can be collected from the patient by spot collection or 24 hour collection.

In a further embodiment of the invention, a biologically active surface comprises an adsorbent comprising silicon dioxide molecules.

A further aspect of the invention includes a kit for diagnosing prostate disease within a subject comprising: a biologically active surface comprising an adsorbent, binding solutions, and instructions to use the kit, wherein the instructions outline a method for diagnosis of a prostate cancer in a subject according to the invention or a method for the differential diagnosis of healthy, non-malignant disease of the prostate, precancerous prostatic lesion, localized cancer of the prostate, metastasised cancer of the prostate, and acute or chronic inflammation of prostatic tissue in a subject according to the invention. In an embodiment, the subject does not have hypertension.

In an embodiment of the invention, a kit comprises a biologically active surface comprising an adsorbent comprised of silicon dioxide molecules.

In an embodiment of the invention, a kit comprises a biologically active surface comprising an adsorbent comprising antibodies specific to PSP94 and PSA.

A further aspect of the invention includes a method for in vitro diagnosis of a prostate cancer in a subject comprising detecting PSP94 and total PSA in a biological sample by: (a) contacting a biological sample from a subject with one or more binding molecule specific for PSP94 and PSA and (b) detecting a quantity, presence or absence of PSP94 and PSA, and determining F/T PSA, in the sample, wherein a quantity, presence or absence of PSP94 and total PSA allows for diagnosis of the subject as healthy or having prostate cancer. In an embodiment, the subject does not have hypertension.

A further aspect of the invention includes a method for in vitro differential diagnosis of prostate cancer and non-malignant disease of the prostate in a subject, comprising detecting PSP94 and total PSA in a biological sample: (a) contacting a biological sample with a binding molecule specific for PSP94 and PSA; and (b) detecting a quantity, presence or absence of PSP94 and PSA, in the sample, wherein the quantity, presence or absence of PSP94 and total PSA allows for the differential diagnosis of the subject as having prostate cancer, and/or having a non-malignant disease of the prostate, or as being healthy. In an embodiment, the subject does not have hypertension.

In an embodiment according to the invention for in vitro diagnosis of prostate cancer in a subject, for in vitro differential diagnosis of prostate cancer and non-malignant disease of the prostate in a subject, or for in vitro differential diagnosis of healthy, prostate cancer, non-malignant disease of the prostate, precancerous prostatic lesion, localized cancer of the prostate, metastasised cancer of the prostate, and acute or chronic inflammation of prostatic tissue in a subject, detection is performed by an immunosorbent assay. In an embodiment, the subject does not have hypertension.

A further aspect of the invention comprises a kit for diagnosis of a prostate disease within a subject comprising a binding solution, one or more binding molecule(s), a detection substrate, and instructions, wherein the instructions outline a method according to the invention for in vitro diagnosis of prostate cancer in a subject, for in vitro differential diagnosis of prostate cancer and non-malignant disease of the prostate in a subject, or for in vitro differential diagnosis of healthy, prostate cancer, non-malignant disease of the prostate, precancerous prostatic lesion, localized cancer of the prostate, metastasised cancer of the prostate, and acute or chronic inflammation of prostatic tissue in a subject. In an embodiment, the subject does not have hypertension.

A further aspect of the invention comprises a use of PSP94 and total PSA for differential diagnosis of non-malignant disease of the prostate, precancerous prostatic lesion, localized cancer of the prostate, metastasised cancer of the prostate or acute or chronic inflammation of prostatic tissue.

A further aspect of the invention comprises a use of the detection or quantification of PSP94 and total PSA in a biological sample from a subject for determination of whether the subject has prostate cancer. In an embodiment, the subject does not have hypertension.

A further aspect of the invention comprises a use of the detection or quantification of PSP94 and total PSA in a biological sample from a subject for determination of whether the subject has non-malignant disease of the prostate. In an embodiment, the subject does not have hypertension.

A further aspect of the invention comprises a use of the detection or quantification of PSP94 and total PSA in a biological sample from a subject for determination of whether the subject has benign prostate disease, precancerous prostatic lesions, localized cancer of the prostate, metastasised cancer of the prostate, or acute or chronic inflammation of the prostate. In an embodiment, the subject does not have hypertension.

A further aspect of the invention comprises a use of PSP94 and total PSA to detect prostate cancer.

A further aspect of the invention includes a method of identifying a molecular entity that inhibits or promotes an activity of PSP94 and total PSA according to the invention comprising: (a) selecting a control animal having PSP94 and total PSA and a test animal having PSP94 and total PSA; (b) treating the test animal using the molecular entity or a library of molecular entities, under conditions to allow specific binding and/or interaction, and (c) determining a relative quantity of PSP94 and total PSA, as between the control animal and the test animal. In an embodiment of the invention, animals are mammals. Mammals may be rats, mice, or primates.

A further aspect of the invention includes a method of identifying a molecular entity that inhibits or promotes an activity of PSP94 and total PSA comprising: (a) selecting a host cell expressing PSP94 and PSA; (b) cloning the host cell and separating the clones into a test group and a control group; (c) treating the test group using the molecular entity or a library of molecular entities under conditions to allow specific binding and/or interaction, and (d) determining a relative quantity of PSP94 and total PSA, as between the test group and the control group.

A further aspect of the invention includes a method for identifying a molecular entity that inhibits or promotes an activity of PSP94 and total PSA comprising: (a) selecting a test group having a host cell expressing PSP94 and total PSA and a control group; (b) treating the test group using the molecular entity or a library of molecular entities; and (c) determining a relative quantity of PSP94 and F/T PSA, as between the test group and the control group.

In an embodiment of the invention, a host cell is a neoplastic or cancer cell.

In an embodiment of any of the methods according to the invention for identifying a molecular entity that inhibits or promotes an activity of PSP94 and total PSA, a library of molecular entities can be nucleotides, oligonucleotides, polynucleotides, amino acids, peptides, polypeptides, proteins, antibodies, immunoglobulins, small organic molecules, pharmaceutical agents, agonists, antagonists, derivatives, and/or combinations thereof.

A further aspect of the invention includes a composition for treating a prostate disease comprising a molecular entity, which modulates PSP94 and total PSA and a pharmaceutically acceptable carrier.

An embodiment of the invention includes a composition for treating a prostate disease selected from the group consisting of prostate cancer and non-malignant disease of the prostate.

A further embodiment includes a composition for treating a prostate disease selected from the group consisting of non-malignant disease of the prostate, precancerous prostatic lesion, localized cancer of the prostate, metastasised cancer of the prostate, and acute or chronic inflammation of prostatic tissue.

A further embodiment of the invention includes a composition comprising a molecular entity that can be polynucleotides, amino acids, polypeptides, small organic molecules, pharmaceutical agents, or combinations thereof. The polypeptides can be antibodies, agonists, antagonists, derivatives, or combinations thereof.

A further aspect of the invention includes a composition for treating prostate disease comprising a molecular entity identified by any one of the methods of invention for identifying a molecular entity, which inhibits or promotes the activity of PSP94 and/or PSA, and a pharmaceutically acceptable carrier.

In an embodiment of the invention, a composition comprises a molecular entity that is comprised of polynucleotides, amino acids, peptides, polypeptides, proteins, small organic molecules, pharmaceutical agents, agonists, antagonists, derivatives or combinations thereof.

A further aspect of the invention includes a use of any composition according to the invention for treating a prostate disease. Prostate disease may be prostate cancer and non-malignant disease of the prostate. The prostate disease may be is selected from the group consisting of non-malignant disease of the prostate, precancerous prostatic lesion, localized cancer of the prostate, metastasised cancer of the prostate, and acute or chronic inflammation of prostatic tissue.

### BRIEF DESCRIPTION OF THE DRAWINGS

FIG. 1 is a flow chart illustrating an example method of diagnosing a subject.

FIG. 2 is a flow chart illustrating an example method of computing a standard score based on at least first and second characteristics of a subject.

FIG. 3 is a schematic block diagram illustrating an architecture of an example computing device for implementing various aspects according to the present disclosure.

FIG. 4 is a ROC Curve for the diagnosis of BPH compared the prostate cancer by PSP94 concentrations in pre-DRE spot urine collection (ng/mL), and PSP94 in 24 hr urine collection (ng/mL).

FIG. 5 is a box and whisker Plot of PSP94 24 hr categorized by Gleason Score. PSP94 in 24 hr collection is in ng/mL and Gleason Score 0=non cancer samples. The upper and lower box is 25% and 75% of data respectively, the line within the box is the median and the whiskers are 5% and 95% limits of the data. Points that are shaded are outliers.

FIG. 6 is a ROC curve for Logistic Regression model for PSP94 spot collection, PSA, PSP94 spot collection for noncancer compared to HGS samples in men without hypertension.

FIG. 7 is a ROC curve for PSP94 spot collection, 24 hr collection and PSA for men without hypertension and PSA values below 10 ng/mL. AUC is 0.923.

### DETAILED DESCRIPTION OF THE INVENTION

The term “biomolecule” refers to a molecule that is produced by a cell or tissue in an organism. Such molecules include, but are not limited to, molecules comprising polynucleotides, amino acids, peptides, polypeptides, proteins, sugars, carbohydrates, fatty acids, lipids, steroids, and combinations thereof (e.g., glycoproteins, ribonucleoproteins, lipoproteins). The terms “nucleotide”, “oligonucleotide” or polynucleotide” refer to DNA or RNA or synthetic origin which may be single-stranded or double-stranded and may represent the sense or the antisense strand. Included as part of the definition of “oligonucleotide” or “polynucleotide” are peptide polynucleotide sequences (i.e. peptide nucleic acids; PNAs), or any DNA-like or RNA-like material (e.g. morpholinos, ribozymes).

“Polypeptide” refers to a peptide or protein containing two or more amino acids linked by peptide bonds, and includes peptides, oligomers, proteins, and the like. Polypeptides can contain natural, modified, or synthetic amino acids. Polypeptides can also be modified naturally, such as by post-translational processing, or chemically, such as amidation acylation, cross-linking, glycosylation, pegylation, and the like.

The terms “antibody” and “immunoglobulin” are used interchangeably in the broadest sense and include monoclonal antibodies (full-length or intact monoclonal antibodies), polyclonal antibodies, humanized, multivalent antibodies, multispecific antibodies (e.g., bispecific antibodies so long as they exhibit the desired biological activity), and antibody fragments.

“Antibody fragments” contain a portion of an intact antibody, generally the antigen binding or variable region of the intact antibody. Examples of antibody fragments include, but are not limited to Fab fragments, Fab′ fragments, Fd′ fragment, Fv fragment, Fd fragment, F(ab′)₂ fragment, dAb fragment, hingeless antibodies, single chain antibodies, diabodies, single arm antigen binding molecules (containing a light chain, a heavy chain and a N-terminally truncated heavy chain constant region sufficient to form a Fc region capable of increasing the half life of the single arm antigen binding molecule), and linear antibodies.

The term “molecular entity” refers to any defined inorganic or organic molecule that is either naturally occurring or is produced synthetically. Such molecules include, but are not limited to, biomolecules as described above, simple and complex molecules, acids and alkalis, alcohols, aldehydes, arenas, amides, amines, esters, ethers, ketones, metals, salts, and derivatives of any of the aforementioned molecules.

The term “fragment” refers to a portion of a polynucleotide or polypeptide sequence that comprises at least 15 consecutive nucleotides or 5 consecutive amino acid residues, respectively. Furthermore, these “fragments” typically retain the biological activity and/or some functional characteristics of the parent polypeptide e.g. antigenicity or structural domain characteristics.

The term “prostatic secretory protein 94” or “PSP94” refers to a 94 amino acid protein secreted by the prostate that functions as a tumor suppressor. PSP94 is the mature protein that is amino acid residues 1 to 94 of the full-length 114 amino acid protein of SEQ ID NO:1. The terms “Prostate Secretory protein 94”, “PSP94”, “Prostate Secreted Seminal Plasma Protein”, “Seminal Plasma Beta-Inhibin”, “Immunoglobulin-binding factor”, “IGBF”, and “PN44” are used interchangeably herein.

The term “PSA” refers to prostate specific antigen. “PSA” is also known as kallikrein III, seminin, semenogelase, γ-seminoprotein and P-30 antigen. PSA is a protein produced by prostate cells that acts as a serine protease. PSA can be found in seminal fluid and prostate tissue. This enzyme can participate in the dissolution of the seminal fluid coagulum and plays an important role in fertility. Some PSA can escape the seminal fluid and be found in the serum. Most PSA in serum is bound to serum proteins, which is referred to as “bound PSA”. PSA serum levels are usually elevated in men with prostate cancer. A serum PSA test is currently the only method used to screen for early detection of prostate cancer. “Free PSA” refers to PSA that is not bound to serum proteins.

The term “total PSA” refers to the sum of free PSA and bound PSA. The terms “biological sample” and “test sample” are used interchangeably and refer to all biological fluids and excretions isolated from any given subject. Such samples include, but are not limited to, blood, blood serum, blood plasma, urine, semen, seminal fluid, seminal plasma, prostatic fluid, pre-ejaculatory fluid (Cowper's fluid), excreta, tears, saliva, sweat, biopsy, ascites, cerebrospinal fluid, lymph, marrow, hair or tissue extract samples such as homogenized tissue, and cellular extracts, and combinations thereof. Tissue samples include samples of tumors.

The term “host cell” refers to a cell that has been transformed or transfected, or is capable of transformation or transfection by an exogenous polynucleotide sequence. It is understood that such terms refer not only to the particular subject cell but also to the progeny or potential progeny of such a cell. Since certain modifications may occur in succeeding generations due to either mutation or environmental influences, such progeny may not, in fact, be identical to the parent cell, but are still included within the scope of the term as used herein.

The term “specific binding” refers to an interaction between two biomolecules that occurs under specific conditions. The binding of two biomolecules is considered to be specific when the interaction between said molecules is substantial. In the context of the invention, a binding reaction is considered substantial when the signal of the peak representing the biomolecule is at least twice that of the signal arising from the coincidental detection of non-biomolecule associated ions in approximately the same mass range, which is the peak as a signal to noise ratio of at least two. Moreover, the phrase “specific conditions” refers to reaction conditions that permit, enable, or facilitate the binding of said molecules such as pH, salt, detergent and other conditions known to those skilled in the art.

The term “interaction” relates to the direct or indirect binding or alteration of biological activity of a biomolecule.

A reagent may be “immobilized” on or in a support by forming a covalent bond between a functional group of the reagent and a reactive group on the surface of the solid phase. In other embodiments, the reagent is “immobilized” on the solid phase by adsorption and ionic binding or may be entrapped in the solid phase, e.g., within cells or lattice type polymers or microcapsules (See Holenberg et al., in Enzymes as Drugs, John Wiley & Sons NY (1981), pages 396-411). The reagent should essentially retain its ability to bind to and/or modify the polypeptide of interest once immobilized to the solid phase.

The term “differential diagnosis” refers to a diagnostic decision between healthy and different disease states, including various stages of a specific disease. A subject is diagnosed as healthy or to be suffering from a specific disease, or a specific stage of a disease based on a set of hypotheses that allow for the distinction between healthy and one or more stages of the disease. A choice between healthy and one or more stages of disease depends on a significant difference between each hypothesis. Under the same principle, a “differential diagnosis” may also refer to a diagnostic decision between one disease type as compared to another (e.g., prostate cancer versus a non-malignant disease of the prostate).

The term “prostate cancer” refers to a malignant neoplasm of the prostate within a given subject, wherein the neoplasm is of epithelial origin and is also referred to as a carcinoma of the prostate. Prostate cancer can be defined according to its type, stage and/or grade. Typical staging systems include the Jewett-Whitmore system and the TNM system (the system adopted by the American Joint Committee on Cancer and the International Union Against Cancer). A typical grading system is the Gleason Score which is a measure of tumour aggressiveness based on pathological examination of tissue biopsy). The term “prostate cancer”, when used without qualification, includes both localized and metastasised prostate cancer. The term “prostate cancer” can be qualified by the terms “localized” or “metastasised” to differentiate between different types of tumour as those words are defined herein. The terms “prostate cancer” and “malignant disease of the prostate” are used interchangeably herein.

The terms “neoplasm” or “tumour” may be used interchangeably and refer to an abnormal mass of tissue wherein the growth of the mass surpasses and is not coordinated with the growth of normal tissue. A neoplasm or tumour may be defined as “benign” or “malignant” depending on the following characteristics: degree of cellular differentiation including morphology and functionality, rate of growth, local invasion and metastasis. A “benign” neoplasm is generally well differentiated, has characteristically slower growth than a malignant neoplasm and remains localised to the site of origin. In addition a benign neoplasm does not have the capacity to infiltrate, invade or metastasise to distant sites. A “malignant” neoplasm is generally poorly differentiated (anaplasia), has characteristically rapid growth accompanied by progressive infiltration, invasion and destruction of the surrounding tissue. Furthermore, a malignant neoplasm has to capacity to metastasise to distant sites.

The term “differentiation” refers to the extent to which parenchymal cells resemble comparable normal cells both morphologically and functionally.

The term “metastasis” refers to spread or migration of cancerous cells from a primary (original) tumour to another organ or tissue, and is typically identifiable by the presence of a “secondary tumour” or “secondary cell mass” of the tissue type of the primary (original) tumour and not of that of the organ or tissue in which the secondary (metastatic) tumour is located. For example, a prostate cancer that has migrated to bone is said to be metastasised prostate cancer, and consists of cancerous prostate cancer cells in the prostate as well as cancerous prostate cancer cells growing in bone tissue.

The terms “a non-malignant disease of the prostate”, “non-prostate cancer state” and “benign prostatic disease” may be used interchangeably and refer to a disease state of the prostate that has not been classified as prostate cancer according to specific diagnostic methods including but not limited to rectal palpitation, PSA scoring, transrectal ultrasonography and tissue biopsy. Such diseases include, but are not limited to, an inflammation of prostatic tissue (i.e., chronic bacterial prostatitis, acute bacterial prostatitis, chronic abacterial prostatitis) and benign prostate hyperplasia.

The term “healthy” refers to an absence of any malignant or non-malignant disease of the prostate; thus, a “healthy individual” may have other diseases or conditions that would normally not be considered “healthy”. A “healthy” individual demonstrates an absence of any malignant or non-malignant disease of the prostate.

The term “pre-cancerous lesion of the prostate” or “precancerous prostate lesion” refers to a biological change within the prostate such that it becomes susceptible to the development of a malignant neoplasm. More specifically, a pre-cancerous lesion of the prostate is a preliminary stage of a prostate cancer. Causes of a pre-cancerous lesion may include, but are not limited to, genetic predisposition and exposure to cancer-causing agents (carcinogens); such cancer causing agents include agents that cause genetic damage and induce neoplastic transformation of a cell.

The term “neoplastic transformation of a cell” refers to an alteration in normal cell physiology and includes, but is not limited to, self-sufficiency in growth signals, insensitivity to growth-inhibitory (anti-growth) signals, evasion of programmed cell death, limitless replicative potential, sustained angiogenesis, and tissue invasion and metastasis.

The term “differentially present” refers to differences in the quantity of a biomolecule present in samples taken from prostate cancer patients as compared to samples taken from subjects having a non-malignant disease of the prostate or healthy subjects. Furthermore, a biomolecule is differentially present between two samples if the quantity of said biomolecule in one sample population is significantly different (defined statistically) from the quantity of said biomolecule in another sample population. For example, a given biomolecule may be present at elevated, decreased, or absent levels in samples of taken from subjects having prostate cancer compared to those taken from subjects who do not have a prostate cancer.

The term “biological activity” may be used interchangeably with the terms “biologically active”, “bioactivity” or “activity” and, for the purposes herein, means an effector or antigenic function that is directly or indirectly performed by a biomarker of the invention (whether in its native or denatured conformation), derivative or fragment thereof. Effector functions include phosphorylation (kinase activity) or activation of other molecules, induction of differentiation, mitogenic or growth promoting activity, signal transduction, immune modulation, DNA regulatory functions and the like. Antigenic functions include possession of an epitope or antigenic site that is capable of cross-reacting with antibodies raised against a naturally occurring or denatured biomarker of the invention, derivative or fragment thereof. Accordingly, a biological activity of such a protein can be that it functions as regulator of a signalling pathway of a target cell. Such a signalling pathway can, for example, modulate cell differentiation, proliferation and/or migration of such a cell, as well as tissue invasion, tumour development and/or metastasis. A target cell according to the invention can be a neoplastic or cancer cell.

The terms “neoplastic cell” and “neoplastic tissue” refer to a cell or tissue, respectively, that has undergone significant cellular changes (transformation). Such cellular changes are manifested by an escape from specific control mechanisms, increased growth potential, alteration in the cell surface, karyotypic abnormalities, morphological and biochemical deviations from the norm, and other attributes conferring the ability to invade, metastasise and kill.

The term “diagnostic assay” can be used interchangeably with “diagnostic method” and refers to the detection of the presence or nature of a pathologic condition. Diagnostic assays differ in their sensitivity and specificity, and their relative usefulness as a diagnostic tool can be measured using ROC-AUC statistics.

Herein, the term “true positives” refers to those subjects having a localized or a metastasised cancer of the prostate or a benign prostate disease, a precancerous prostatic lesion, or an acute or a chronic inflammation of prostatic tissue and who are categorized as such by the diagnostic assay. Depending on context, the term “true positives” may also refer to those subjects having either prostate cancer or a non-malignant disease of the prostate, who are categorized as such by the diagnostic assay.

Herein, the term “false negatives” refers to those subjects having either a localized or a metastasised cancer of the prostate, a benign prostate disease, a precancerous prostatic lesion, or an acute or a chronic inflammation of prostatic tissue and who are not categorized as such by the diagnostic assay. Depending on context, the term “false negatives” may also refer to those subjects having either prostate cancer or a non-malignant disease of the prostate and who are not categorized as such by the diagnostic assay.

Herein, the term “true negatives” refers to those subjects who do not have a localized or a metastasised cancer of the prostate, a benign prostate disease, a precancerous prostatic lesion, or an acute or a chronic inflammation of prostatic tissue and who are categorized as such by the diagnostic assay. Depending on context, the term “true negatives” may also refer to those subjects who do not have prostate cancer or a non-malignant disease of the prostate and who are categorized as such by the diagnostic assay.

Herein, the term “false positives” refers to those subjects who do not have a localized or a metastasised cancer of the prostate, a benign prostate disease, a precancerous prostatic lesion, or an acute or a chronic inflammation of prostatic tissue but are categorized by the diagnostic assay as having a localized or metastasised cancer of the prostate, a benign prostate disease, a precancerous prostatic lesion or an acute or chronic inflammation of prostatic tissue. Depending on context, the term “false positives” may also refer to those subjects who do not have prostate cancer or a non-malignant disease of the prostate but are categorized by the diagnostic assay as having prostate cancer or a non-malignant disease of the prostate.

The term “sensitivity”, as used herein in the context of its application to diagnostic assays, refers to the proportion of all subjects with localized or metastasised cancer of the prostate, a benign prostate disease, a precancerous prostatic lesion, or an acute or a chronic inflammation of prostatic tissue that are correctly identified as such (that is, the number of true positives divided by the sum of the number of true positives and false negatives).

The term “specificity” of a diagnostic assay, as used herein in the context of its application to diagnostic assays, refers to the proportion of all subjects with neither localized or metastasised cancer of the prostate nor a benign prostate disease, a precancerous prostatic lesion, or an acute or a chronic inflammation of prostatic tissue that are correctly identified as such (that is, the number of true negatives divided by the sum of the number of true negatives and false positives).

The term “adsorbent” refers to any material that is capable of accumulating (binding) a given biomolecule. The adsorbent typically coats a biologically active surface and is composed of a single material or a plurality of different materials that are capable of binding a biomolecule. Such materials include, but are not limited to, anion exchange materials, cation exchange materials, metal chelators, polynucleotides, oligonucleotides, peptides, antibodies, naturally occurring compounds, synthetic compounds, etc.

The phrase “biologically active surface” refers to any two- or three-dimensional extensions of a material that biomolecules can bind to, or interact with, due to the specific biochemical properties of this material and those of the biomolecules. Such biochemical properties include, but are not limited to, ionic character (charge), hydrophobicity, or hydrophilicity.

The phrase “binding biomolecule” refers to a molecule that displays an affinity for another biomolecule.

The term “immunogen” may be used interchangeably with the phrase “immunising agent” and refers to any substance or organism that provokes an immune response when introduced into the body of a given subject. All immunogens are considered as antigens and, in the context of the invention, can be defined on the basis of their immunogenicity, wherein “immunogenicity” refers to the ability of the immunogen to induce either a humoral or a cell-mediated immune response. In the context of the invention an immunogen that induces a “humoral immune response” activates antibody production and secretion by cells of the B-lymphocyte lineage (B-cells) and thus can be used to for antibody production as described herein. Such immunogens may be polysaccharides, proteins, lipids or nucleic acids, or they may be lipids or nucleic acids that are complexed to either a polysaccharide or a protein.

The term “solution” refers to a homogeneous mixture of two or more substances. Solutions may include, but are not limited to buffers, substrate solutions, elution solutions, wash solutions, detection solutions, standardisation solutions, chemical solutions, solvents, etc.

The phrase “coupling buffer” refers to a solution that is used to promote covalent binding of biomolecules to a biological surface.

The phrase “blocking buffer” refers to a solution that is used to (prevent) block unbound binding sites of a given biological surface from interacting with biomolecules in an unspecific manner.

The term “chromatography” refers to any method of separating biomolecules within a given sample such that the original native state of a given biomolecule is retained. Separation of a biomolecule from other biomolecules within a given sample for the purpose of enrichment, purification and/or analysis, may be achieved by methods including, but not limited to, size exclusion chromatography, ion exchange chromatography, hydrophobic and hydrophilic interaction chromatography, metal affinity chromatography, wherein “metal” refers to metal ions (e.g. nickel, copper, gallium, zinc, iron or cobalt) of all chemically possible valences, or ligand affinity chromatography wherein “ligand” refers to binding molecules, preferably proteins, antibodies, or DNA. Generally, chromatography uses biologically active surfaces as adsorbents to selectively accumulate certain biomolecules.

The phrase “mass spectrometry” refers to a method comprising employing an ionisation source to generate gas phase ions from a biological entity of a sample presented on a biologically active surface, and detecting the gas phase ions with an ion detector. Comparison of the time the gas phase ions take to reach the ion detector from the moment of ionisation with a calibration equation derived from at least one molecule of known mass allows the calculation of the estimated mass to charge ratio of the ion being detected.

The phrases “mass to charge ratio”, “m/z ratio” or “m/z” can be used interchangeably and refer to the ratio of the molecular weight (grams per mole) of an ion detected by mass spectrometry to the number of charges the ion carries. Thus a single biomolecule can be assigned more than one mass to charge ratio by a mass spectrometer if that biomolecule can be ionised into more than one species each of which carries a different number of charges.

The terms “detect”, “detection” or “detecting” refer to the identification of the presence, absence, or quantity of a given biomolecule.

The acronym “ROC-AUC” refers to the area under a receiver operator characteristic curve. This is a widely accepted measure of diagnostic utility of some tool, taking into account both the sensitivity and specificity of the tool. Typically, ROC-AUC ranges from 0.5 to 1.0, where a value of 0.5 indicates the tool has no diagnostic value and a value of 1.0 indicates the tool has 100% sensitivity and 100% specificity.

The term “hypertension” refers to elevated blood pressure. A definition of hypertension is arbitrary because the risk of cardiovascular disease related to blood pressure level increases steadily across the spectrum of blood pressure values, however optimum blood pressure is defined as less than 115/75 mm Hg. Normal blood pressure (the level associated with minimal risk) for adults 18 years of age or older is a systolic blood pressure of less than 120 mm Hg and a diastolic blood pressure of less than 80 mm Hg. For the general population, hypertension is defined as a systolic BP of 140 mm Hg or higher or a diastolic BP of 90 mm Hg or higher. (Lewington S, Clarke R, Qizilbash N, et al: Age-specific relevance of usual blood pressure to vascular mortality: a meta-analysis of individual data from one million adults in 61 prospective studies. Lancet 360:1903, 2002). The term “sensitivity” refers to the proportion of patients with the outcome in whom the results of the decision rule are abnormal. Typically, the outcome is disadvantageous to the patient. The term “specificity” refers to the proportion of patients without the outcome in whom the results of the decision rule are normal.

It is to be understood that the present invention is not limited to the particular materials and methods described or equipment, as these may vary. It is also to be understood that the terminology used herein is for the purpose of describing particular embodiments only, and is not intended to limit the scope of the present invention, which will be limited only by the appended claims.

It should be noted that as used herein and in the appended claims, the singular forms “a,” “an,” and “the” include plural reference unless the context clearly dictates otherwise. Thus, for example, a reference to “an antibody” is a reference to one or more antibodies and derivatives thereof known to those skilled in the art, and so forth.

PSP94

PSP94 is a versatile protein that plays a role in several biological processes within the reproductive tract ranging from modulating the circulation of follicle-stimulating hormone (FSH) to inducing apoptosis in prostate cancer cells (Sheth et al., 1984; Chao et al., 1996; Hirano et al., 1996; Garde et al., 1999; Shukeir et al., 2003). It is one of the three major proteins secreted by the normal human prostate gland. As a secreted protein, this molecule is found in a variety of bodily fluids including serum (Teni et al., 1988; Reeves et al., 2005; van Huizen et al., 2005), urine (Teni et al., 1988; Liu et al., 1993), seminal plasma fluid (Sheth et al., 1984; Dube et al., 1987; von der Kammer et al., 1991) and mucous gland secretions (Weiber et al., 1990). PSP94 occurs in both the free and bound forms in serum (Wu et al., 1999).

Full-length PSP94 has the following sequence:
(SEQ ID NO: 1; Accession No. AB29732.1/GI:460569)
MNVLLGSVVIFATFVTLCNASCYFIPNEGVPGDSTRKCMDLKGNKHPINS
EWQTDNCETCTCYETEISCCTLVSTPVGYDKDNCQRIFKKEDCKYIVVEK
KDPKKTCSVSEWII
Mature PSP94 has the following sequence:
(SEQ ID NO: 2)
SCYFIPNEGVPGDSTRKCMDLKGNKHPINSEWQTDNCETCTCYETEISCC
TLVSTPVGYDKDNCQRIFKKEDCKYIVVEKKDPKKTCSVSEWII

### Diagnostic Tools

Although PSP94 has been shown to be a useful discriminatory factor for diagnosis and/or prognosis of prostate cancer, diagnostic tools utilizing this protein are both invasive and lacking sensitivity. A diagnostic tool utilising PSP94 and total PSA has not yet been described. This improves the discriminatory value for prostate cancer over each of the markers when used alone. In addition to this, urine samples are the preferred samples for diagnostic tools described herein, making the test ideal for clinical application. Embodiments of the invention are non-invasive and cost-effective.

The present invention relates to methods for differential diagnosis of prostate cancer or a non-malignant disease of the prostate by detecting PSP94 and total PSA, within a biological sample of a given subject, comparing results with samples from healthy subjects, subjects having a non-malignant disease of the prostate and subjects having prostate cancer, wherein the comparison allows for the differential diagnosis of a subject as healthy, having non-malignant disease of the prostate or having prostate cancer.

One aspect of the invention includes a method for diagnosing prostate cancer in a subject comprising: (a) detecting a quantity, presence or absence of PSP94 and total PSA; and (b) classifying the subject as having or not having prostate cancer.

In an embodiment of the invention, the step of classifying the subject comprises comparing the quantity, presence or absence of PSP94 and total PSA, with a reference value indicative of a prostate cancer. The reference value comprises PSP94 and total PSA. A further aspect of the invention includes a method for differential diagnosis of prostate cancer and non-malignant disease of the prostate in a subject, comprising: (a) detecting a quantity, presence or absence of PSP94 and total PSA, in a biological sample; and (b) classifying the subject as having prostate cancer, non-malignant disease of the prostate, or as healthy, based on the quantity, presence or absence of PSP94 and total PSA.

In an embodiment of the invention, the step of classifying the subject comprises comparing the quantity, presence or absence of PSP94 and total PSA, with a reference value indicative of prostate cancer and a reference value indicative of a non-malignant disease of the prostate. Reference values comprise PSP94 and total PSA, characterised as being diagnostic for prostate cancer or for a non-malignant disease of the prostate.

A further aspect of the invention includes a method for differential diagnosis of healthy, non-malignant disease of the prostate, precancerous prostatic lesion, localized cancer of the prostate, metastasised cancer of the prostate, and acute or chronic inflammation of prostatic tissue in a subject, comprising: (a) detecting a quantity, presence or absence of PSP94 and total PSA, in a biological sample; and (b) classifying the subject as having non-malignant disease of the prostate, precancerous prostate lesion, localized cancer of the prostate, metastasised cancer of the prostate, and/or acute or chronic inflammation of prostatic tissue, or as healthy, based on the quantity, presence or absence of PSP94 and total PSA, in the biological sample. Reference values may include values for good health, non-malignant disease of the prostate, precancerous prostate lesion, localized cancer of the prostate, metastasised cancer of the prostate, and/or acute or chronic inflammation of prostatic tissue.

In one embodiment, a biologically active surface comprises an adsorbent comprising silicon dioxide molecules. In another embodiment, a biologically active surface comprises an adsorbent comprised of antibodies. Antibodies may be antibodies specific to PSP94 and PSA. Biologically active surfaces useful for practicing the methods of the invention are further described in greater detail below.

In addition, other methods of determining a quantity, presence or absence of PSP94 and total PSA, in a biological sample can be utilized, such as ELISA utilizing antibodies targeted to a biomarker of the invention. In any of the embodiments of the methods described above, PSP94 and total PSA, may be detected within a given biological sample. Detection of biomolecules of the invention is based on specific sample pre-treatment conditions, the pH of binding conditions, the adsorbent used on the biologically active surface. In one embodiment of the invention, a biomolecule of the invention can include PSP94 and total PSA, and may be used to diagnose a subject as being healthy, having a non-malignant disease of the prostate, having a precancerous prostatic lesion, having a localized cancer of the prostate, having a metastasised cancer of the prostate, or having an acute or a chronic inflammation of prostatic tissue. In another embodiment of the invention, PSP94 and total PSA may be used in combination with at least one other biomolecule to diagnose a subject as being healthy, having of a non-malignant disease of the prostate, having a precancerous prostatic lesion, having a localized cancer of the prostate, having a metastasised cancer of the prostate, or having an acute or a chronic inflammation of prostatic tissue.

In yet another embodiment of the invention, detection and/or quantification of biomolecules, including PSP94 and total PSA, may be used in combination with another diagnostic tool to diagnose a subject as being healthy, having a non-malignant disease of the prostate, having a precancerous prostatic lesion, having a localized cancer of the prostate, having a metastasised cancer of the prostate, or having an acute or a chronic inflammation of prostatic tissue. For example, PSP94 and total PSA may be used in combination with at least one other diagnostic tool specific for prostate cancer detection such as, but not limited to, DRE, rectal palpitation, biopsy evaluation using Gleason scoring, radiography, proteo-imaging, and symptomological evaluation by a qualified clinician. In another embodiment, PSP94 and total PSA may also be combined with the detection of PCA3 (also known as DD3) and/or a prostate disease specific microRNA pattern.

Methods for detecting biomolecules according to the invention have many applications. For example, PSP94 and total PSA can be measured to differentiate between healthy subjects, subjects having a non-malignant disease of the prostate, subjects having a precancerous prostatic lesion, subjects having a localized cancer of the prostate, subjects having a metastasised cancer of the prostate, or subjects with an acute or a chronic inflammation of prostatic tissue, and thus are useful as an aid in diagnosis of a non-malignant disease of the prostate, a precancerous prostatic lesion, or a localized cancer of the prostate, a metastasised cancer of the prostate, or an acute or a chronic inflammation of prostatic tissue. Alternatively, said biomolecules may be used to diagnose a subject as being healthy.

Another aspect of the invention includes an in vitro method for diagnosis of prostate cancer in a subject comprising detecting differentially expressed biomarkers in a biological sample by: (a) contacting a sample with a binding molecule specific for PSP94 and a second binding molecule specific for PSA, and (b) detecting a quantity, presence or absence of PSP94 and total PSA, wherein the quantity, presence, or absence of PSP94 and total PSA, and (c) diagnosing the subject as healthy or having prostate cancer.

A further aspect of the invention includes a method for in vitro differential diagnosis of prostate cancer and non-malignant disease of the prostate in a subject, comprising detecting one or more differentially expressed biomarkers in a biological sample: (a) contacting a sample with a first binding molecule specific for PSP94 and a second binding molecule specific for PSA, (b) detecting a quantity, presence or absence of PSP94 and total PSA in the sample, and (c) diagnosing the subject as having prostate cancer, and/or having a non-malignant disease of the prostate, or as being healthy.

Still a further aspect of the invention includes an in vitro method for differential diagnosis of healthy, prostate cancer, non-malignant disease of the prostate, precancerous prostatic lesion, localized cancer of the prostate, metastasised cancer of the prostate, and acute or chronic inflammation of prostatic tissue in a subject, comprising detection of PSP94 and total PSA, in a biological sample by: (a) contacting a sample with a first binding molecule specific for PSP94 and a second binding molecule specific for PSA, and (b) detecting a quantity, presence or absence of PSP94 and total PSA, wherein the presence or absence of PSP94 and total PSA, allows for the differential diagnosis of the subject as healthy, having non-malignant disease of the prostate, precancerous prostate lesions, localized cancer of the prostate, metastasised cancer of the prostate, and/or having acute or chronic inflammation of the prostate, or as being healthy.

Binding molecules include, but are not limited to polynucleotides, polypeptides (e.g., monoclonal and/or polyclonal antibodies, antigens, etc.), carbohydrates (e.g., sugars), fatty acids, lipids, steroids, or combinations thereof (e.g. glycoproteins, ribonucleoproteins, lipoproteins), compounds or synthetic molecules. In one preferred embodiment, binding molecules are antibodies specific for PSP94 or PSA. For example, antibodies or fragments thereof may be utilized for the detection of PSP94 and PSA. Depending on the nature of a biological sample, it is possible to determine not only presence of a biomolecule, but also its cellular distribution. For example, in a blood serum sample, only serum levels of a given biomolecule can be detected, whereas its level of expression and cellular localisation can be detected in histological samples.

In another example, an antibody directed against a biomolecule of the invention that is coupled to an enzyme can be detected using a chromogenic substrate that can be recognized and cleaved by the enzyme to produce a chemical moiety, which is readily detected using spectrometric, fluorimetric or visual means. Enzymes used for labelling include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. Detection may also be accomplished by visual comparison of the extent of the enzymatic reaction of a substrate with that of similarly prepared standards. Alternatively, radio-labelled antibodies can be detected using a gamma or a scintillation counter, or they can be detected using autoradiography. In another example, fluorescently labelled antibodies are detected based on the level at which the attached compound fluoresces following exposure to a given wavelength. Fluorescent compounds typically used in antibody labelling include, but are not limited to, fluorescein isothiocynate (FITC), rhodamine, phycoerthyrin, phycocyanin, allophycocyani, o-phthaldehyde and fluorescamine. In yet another example, antibodies coupled to a chemi- or bioluminescent compound can be detected by determining the presence of luminescence. Such compounds include, but are not limited to, luminal, isoluminal, theromatic acridinium ester, imidazole, acridinium salt, oxalate ester, luciferin, luciferase and aequorin.

Furthermore, in vivo techniques for detecting a biomolecule include introducing into a subject a labelled antibody directed against a biomolecule, which can be PSP94 and/or PSA.

In addition, methods of the invention for differential diagnosis of healthy subjects, subjects having a non-malignant disease of the prostate, subjects having a precancerous prostatic lesion, subjects having a localized cancer of the prostate, subjects having a metastasised cancer of the prostate and/or subjects having an acute or chronic inflammation of prostatic tissue, described herein may be combined with other diagnostic methods to improve the outcome of the differential diagnosis.

Methods of the invention can also be used for differential diagnosis of healthy subjects, subjects having a precancerous prostatic lesions, subjects having a non-malignant disease of the prostate, subjects having a localized cancer of the prostate, subjects having metastasised cancer of the prostate, and/or subjects having acute or chronic inflammation of the prostate, or any two or more of the above states.

### Biological Samples of the Invention

Typically, PSP94 and PSA are detected in urine samples, but their detection is not limited to urine samples. Biomolecules of the invention can be detected in blood, blood serum, blood plasma, urine, semen, seminal fluid, seminal plasma, prostatic fluid, pre-ejaculatory fluid (Cowper's fluid), excreta, tears, saliva, sweat, biopsy, ascites, cerebrospinal fluid, lymph, or tissue extract (biopsy) samples. Preferably, biological samples used to detect biomolecules of the invention are urine, semen, seminal fluid, seminal plasma, prostatic fluid, or pre-ejaculatory fluid (Cowper's fluid). Furthermore, biological samples can be isolated from mammalian subjects, preferably humans.

A subject that is said to have a prostate cancer possesses morphological, biochemical and functional alterations of their prostatic tissue such that the tissue can be characterised as a malignant neoplasm. The stage to which a prostate cancer has progressed can be determined using known methods currently available to those skilled in the art (e.g., Union Internationale Contre Cancer (UICC) system or American Joint Committee on Cancer (AJC)). Currently, the most widely used method for determining the extent of malignancy of a prostatic neoplasm is the Gleason Grading system. Gleason grading is based exclusively on the architectural pattern of the glands of a prostatic neoplasm, wherein the ability of neoplastic cells to structure themselves into glands resembling those of the normal prostate is evaluated using a scale of 1 to 5. For example, neoplastic cells that are able to architecturally structure themselves such that they resemble normal prostate gland structure are graded 1-2, whereas neoplastic cells that are unable to do so are graded 4-5. As known to those skilled in the art, a prostatic neoplasm whose tumour structure is nearly normal will tend to behave, biologically, as normal tissue and therefore it is unlikely that it will be aggressively malignant. Gleason score may be integrated with other grading methods and/or staging systems to determine cancer stage.

A subject is said to have a non-malignant disease of the prostate possesses morphological and/or biochemical alterations of their prostatic tissue but does not exhibit malignant neoplastic properties known to those skilled in the art. Such diseases include, but are not limited to, inflammatory and proliferative lesions, as well as benign disorders of the prostate. Within the context of the invention, inflammatory lesions encompass acute and chronic bacterial prostatitis, as well as chronic abacterial prostatitis, proliferative lesions include benign prostate hyperplasia (BPH).

### Biologically Active Surfaces

Biologically active surfaces include, but are not limited to, surfaces that contain adsorbents with anion exchange properties (adsorbents that are positively charged), cation exchange properties (adsorbents that are negatively charged), hydrophobic properties, reverse phase chemistry, groups such as nitriloacetic acid that immobilize metal ions such as nickel, gallium, copper, or zinc (metal affinity interaction), or biomolecules such as proteins, antibodies, nucleic acids, or protein binding sequences, covalently bound to the surface via carbonyl diimidazole moieties or epoxy groups (specific affinity interaction).

These surfaces may be located on matrices like polysaccharides such as sepharose, e.g. anion exchange surfaces or hydrophobic interaction surfaces, or solid metals, e.g. antibodies coupled to magnetic beads or a metal surface. Surfaces may also include gold-plated surfaces such as those used for Biacore™ Sensor Chip technology.

Biologically active surfaces are able to adsorb biomolecules like nucleotides, polynucleotides, amino acids, polypeptides (e.g., monoclonal and/or polyclonal antibodies), steroids, carbohydrates (e.g., sugars), fatty acids, lipids, hormones, and combinations thereof (e.g., glycoproteins, ribonucleoproteins, lipoproteins).

Devices that use biologically active surfaces to selectively adsorb biomolecules can be chromatography columns for Fast Protein Liquid Chromatography (FPLC) and High Pressure Liquid Chromatography (HPLC), where the matrix, e.g. a polysaccharide, carrying the biologically active surface, is filled into vessels (usually referred to as “columns”) made of glass, steel, or synthetic materials like polyetheretherketone (PEEK).

In yet another embodiment, devices that use biologically active surfaces to selectively adsorb biomolecules may be metal strips carrying thin layers of a biologically active surface on one or more spots of the strip surface to be used as probes for gas phase ion spectrometry analysis, for example the PS20 ProteinChip® array for (Ciphergen Biosystems, Inc.) for surface-enhanced laser desorption/ionization (SELDI) analysis.

Detection of Biomolecules of the Invention

In another aspect of the invention, biomolecules (e.g., PSP94 and total PSA) can be detected using other known methods. For example, an in vitro binding assay can be used to detect a biomolecule within a biological sample of a given subject. A given biomolecule can be detected within a biological sample by contacting the biological sample from a given subject with specific binding molecule(s) under conditions conducive for an interaction between the given binding molecule(s) and a biomolecule. Binding molecules include, but are not limited to, nucleic acids, nucleotides, polynucleotides, amino acids, polypeptides (e.g., monoclonal and/or polyclonal antibodies, and antigens), carbohydrates (e.g., sugars), fatty acids, lipids, steroids, or combinations thereof (e.g. glycoproteins, ribonucleoproteins, lipoproteins), compounds or synthetic molecules. Preferably, binding molecules are antibodies specific for PSP94 or PSA. Biomolecules detected using the above-mentioned binding molecules include, but are not limited to, molecules comprising nucleic acids, nucleotides, polynucleotides, amino acids, polypeptides (e.g., monoclonal and/or polyclonal antibodies, antigens), carbohydrates (e.g., sugars), fatty acids, lipids, steroids, and combinations thereof (e.g., glycoproteins, ribonucleoproteins, lipoproteins).

### Sandwich Assay

Sandwich assays for detecting a biomolecule, which can be PSP94 and/or PSA, can be used as a diagnostic tool for diagnosis of a subject as being healthy, having a non-malignant disease of the prostate, having a precancerous prostatic lesion, having a localized cancer of the prostate, or a metastasised cancer of the prostate, or having an acute or a chronic inflammation of prostatic tissue. In the context of the invention, sandwich assays comprise attaching a monoclonal antibody to a solid surface such as a plate, tube, bead, or particle, wherein the antibody is preferably attached to the well surface of a 96-well microtitre plate. A pre-determined volume of sample (e.g., serum, urine, tissue cytosol) containing a subject biomarker is added to the solid phase antibody, and the sample is incubated for a period of time at a pre-determined temperature conducive for specific binding of subject biomarkers within the given sample to the solid phase antibody. Following incubation, the sample fluid is discarded, and the solid phase is washed with buffer to remove any unbound material. A second monoclonal antibody (to a different determinant on the subject biomarker) is added to the solid phase. This antibody is labelled with a detector molecule or atom (e.g., enzyme, fluorophore, chromophore, or ¹²⁵I), and the solid phase is incubated with the second antibody. The second antibody is decanted and the solid phase is washed with buffer to remove unbound material.

The amount of bound label, which is proportional to the amount of subject biomarker present in the sample, can be quantitated.

### Kits

A further aspect of the invention comprises a kit for diagnosing a prostate disease within a subject comprising: a biologically active surface comprising an adsorbent, binding solutions, and instructions to use the kit, wherein the instructions outline a method for diagnosis of a prostate cancer in a subject or a method for differential diagnosis of healthy, non-malignant disease of the prostate, precancerous prostatic lesion, localized cancer of the prostate, metastasised cancer of the prostate, and acute or chronic inflammation of prostatic tissue in a subject according to the invention.

Any of the biologically active surfaces described herein may be used to practice the invention. In an embodiment of the invention, a biologically active surface may comprise an adsorbent comprising of silicon dioxide molecules. In another embodiment of the invention, a biologically active surface may comprise an adsorbent comprising antibodies specific to PSP94 and total PSA.

A further aspect of the invention comprises a kit for diagnosing prostate disease within a subject comprising a binding solution, a binding molecule, a detection substrate, and instructions, wherein the instructions describe an in vitro method for diagnosis of a prostate cancer in a subject, an in vitro method for differential diagnosis of prostate cancer and non-malignant disease of the prostate in a subject, or an in vitro method for differential diagnosis of healthy, prostate cancer, non-malignant disease of the prostate, precancerous prostatic lesion, localized cancer of the prostate, metastasised cancer of the prostate, and acute or chronic inflammation of prostatic tissue in a subject.

Yet another aspect of the invention comprises kits using methods of the invention as described in another section for differential diagnosis of prostate cancer or a non-malignant disease of the prostate, wherein the kits are used to detect biomolecules, which can be PSP94 and total PSA.

Methods used to detect biomolecules, which can be PSP94 and total PSA can also be used to determine whether a subject is at risk of developing prostate cancer or has developed prostate cancer. Such methods may also be employed in the form of a diagnostic kit comprising a binding molecule specific to a biomolecule, which can be PSP94 and PSA, solutions and materials necessary for the detection of a biomolecule of the invention, and instructions to use the kit based on the above-mentioned methods.

For example, a kit can be used to detect biomolecules such as PSP94 and PSA and have many applications. For example, kits can be used to differentiate whether a subject is healthy, has a non-malignant disease of the prostate, or a prostate cancer, thus aiding diagnosis of a prostate cancer and/or a non-malignant disease of the prostate. Moreover, kits can be used to differentiate whether a subject is healthy, having a non-malignant disease of the prostate, has a precancerous prostatic lesion, has a localized cancer of the prostate, has a metastasised cancer of the prostate, or has an acute or a chronic inflammation of the prostate.

In an embodiment, a kit may comprise instructions on how to use the kit, a biologically active surface comprising an adsorbent, wherein the adsorbent is suitable for binding one or more biomolecules of the invention, a denaturation solution for the pre-treatment of a sample, a binding solution, and one or more washing solution(s) or instructions for making a denaturation solution, binding solution, or washing solution(s), wherein the combination of solutions allows for the detection of a biomolecule using gas phase ion spectrometry. Such kits can be prepared from materials described in other previously detailed sections (e.g., denaturation buffer, binding buffer, adsorbents, washing solution(s), etc.).

In another embodiment, a kit may comprise a first substrate comprising an adsorbent thereon (e.g., a particle functionalised with an adsorbent) and a second substrate onto which the first substrate can be positioned to form a probe, which is removably insertable into a gas phase ion spectrometer. In other embodiments, a kit may comprise a single substrate, which is in the form of a removably insertable probe with adsorbents on the substrate.

In another embodiment, a kit may comprise a binding molecule(s) that specifically binds to a biomolecule, which can be PSP94 and/or PSA, a detection reagent, appropriate solutions and instructions on how to use the kit. Such kits can be prepared from materials described above and known materials. A binding molecule used within such a kit may include, but is not limited to, nucleic acids, nucleotides, polynucleotides, amino acids, polypeptides (e.g., monoclonal and/or polyclonal antibodies), carbohydrates (e.g., sugars), fatty acids, lipids, steroids, hormones, or a combination thereof (e.g. glycoproteins, ribonucleoproteins, lipoproteins), compounds or synthetic molecules. In another embodiment, a kit comprises a binding molecule or panel of binding molecules that specifically bind to PSP94 and/or PSA, a detection reagent, appropriate solutions and instructions on how to use the kit. Each binding molecule would be distinguishable from every other binding molecule in a panel of binding molecules, yielding easily interpreted signal for each of the biomolecules detected by the kit. Such kits can be prepared from the materials described above and known materials. A binding molecule can include, but is not limited to, nucleic acids, nucleotides, polynucleotides, amino acids, polypeptides (e.g., monoclonal and/or polyclonal antibodies), carbohydrates (e.g., sugars), fatty acids, lipids, steroids, hormones, or a combination thereof (e.g. glycoproteins, ribonucleoproteins, lipoproteins), compounds or synthetic molecules.

In any of the embodiments described above, a kit may optionally further comprise a standard or control biomolecule so that the biomolecules detected within the biological sample can be compared with said standard to determine if the test amount of a marker detected in a sample is a diagnostic amount consistent with a diagnosis of a non-malignant disease of the prostate, a precancerous prostatic lesion, localized cancer of the prostate, metastasised cancer of the prostate, acute or a chronic inflammation of the prostate. Likewise, a biological sample can be compared with said standard to determine if the test amount of a marker detected is said sample is a diagnostic amount consistent with a diagnosis as healthy.

Patients with hypertension can have higher PSP94 values, even in the absence of non-malignant prostate disease or prostate cancer. In any of the embodiments described above, a method of diagnosis includes methods where a subject with hypertension is excluded from said method. Additionally, any database entries of the described embodiments and/or reference values can be obtained from a population of subjects, wherein the population of subjects excludes subjects with hypertension.

### Exemplary Methods and Systems for Performing Same

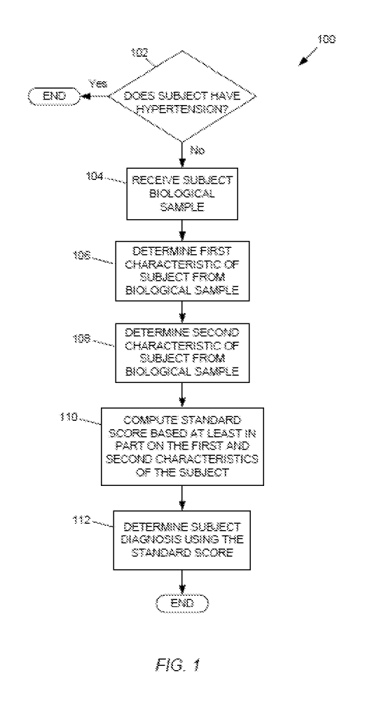

FIG. 1 is a flow chart illustrating an example method 100 of diagnosing a subject. In an example embodiment, method 100 includes operations 102, 104, 106, 108, 110, and 112.

Operation 102 is performed to determine whether the subject has hypertension. If so, method 100 ends. If not, operation 100 proceeds to operation 104.

In an embodiment, operation 102 includes entering a value to correspond that a subject has been diagnosed with hypertension by a physician. In some example embodiments, operation 102 includes measuring the subject's blood pressure, or otherwise determining the subject's blood pressure (such as by retrieving it from the patient's medical record or receiving it from a blood pressure measurement device). In some embodiments, the blood pressure is measured using a blood pressure measurement device, such as a sphygmomanometer. In some embodiments the blood pressure measurement device includes an inflatable cuff attached to a mercury or aneroid manometer. In another embodiment, the blood pressure measurement device is an ambulatory blood pressure monitor. In some embodiments, the blood pressure measurement device is a digital device, such as including a processing device and a memory device. Some embodiments further include a data communication device configured to communicate digital data to another computing device. Some embodiments include at least one processing device and at least one memory device. Some embodiments include programmable electronics including at least one processing and at least one memory device.

Once the blood pressure has been measured, operation 102 determines whether the subject has hypertension, such as by comparing the blood pressure to a threshold value. In some embodiments, the subject's age is also considered. A lookup table stored in a memory device is used in some embodiments to determine whether the subject has hypertension based at least in part on the measured blood pressure, and in some embodiments, other factors, such as the subject's age.

In operation 104, a subject biological sample is received. An example of a biological sample is a urine sample. Examples of possible urine samples include spot collection urine samples and 24 hour collection urine samples. In another embodiment, a biological sample is a blood sample, such as including blood serum. In some embodiments, multiple biological samples are used, such as a urine sample and a blood sample. Other embodiments include other biological samples or combinations of biological samples, such as those discussed herein or other biological samples. The biological samples can be at least temporarily stored in a container (e.g., vial, syringe, cup, etc.).

Following operation 104, the subject biological sample is evaluated through operations 106 and 108, which can be performed in any sequence or simultaneously. In operation 106, the subject biological sample is evaluated to determine a first characteristic of the subject. An example of a first characteristic is a PSA value. In operation 108, the subject biological sample is evaluated to determine a second characteristic of the subject. An example of a second characteristic is a PSP94 value.

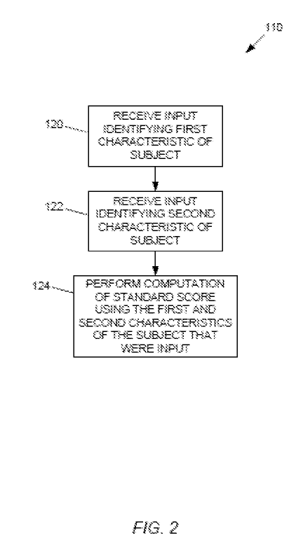

Once the first and second characteristics of the subject have been determined, operation 110 is performed to compute a standard score based at least in part on the first and second characteristics of the subject. An example of operation 110 is illustrated and described herein with reference to FIG. 2.

In some embodiments, once the standard score has been computed, operation 112 is performed to diagnose the subject using the standard score. For example, the standard score is compared to two or more possible ranges of scores to determine which of the ranges the standard score falls within. Once that range has been identified, the diagnosis is determined by identifying a diagnosis associated with the identified range of scores. In some embodiments, one or more cutoff values are used to identify boundaries of the ranges. For example, if the standard score is less than the cutoff value, the diagnosis is negative, while if the standard score is greater than or equal to the cutoff value, the diagnosis is positive.

Operation 112 is not performed in all embodiments. For example, some embodiments are a method of generating a standard score. In some embodiments, the standard score is computed in operation 110 and is then saved to a memory device. In some embodiments the standard score is displayed on a display device. In other embodiments, the standard score is communicated to a computing device, such as across a network. In some embodiments the standard score is stored in the subject's medical record in a medical records database. In yet further embodiments, the standard score is stored in a database where it is compiled with the standard scores of other subjects for further data processing and analysis.

FIG. 2 is a flow chart illustrating an example method of computing a standard score based at least in part on first and second characteristics of a subject, as illustrated and described herein as operation 110, shown in FIG. 1. However, in some embodiments the method shown in FIG. 2 is performed independent from method 100, shown in FIG. 1. In some embodiments, operation 110 includes operations 122, 124, and 126.

Operations 122 and 124 are first performed to receive inputs identifying first and second characteristics of the subject. In some embodiments the inputs are provided by a human, such as a caregiver or the subject, through a user input device. Examples of user input devices include a keyboard, a mouse, a voice recognition system, a touch display, and other user input devices. A computing device operates to prompt the user for the inputs in some embodiments.

In yet an embodiment, the inputs are retrieved from a memory device. An example of a memory device is a database. An example of a database is a medical record of the subject.

The inputs identifying the first and second characteristics are typically stored in one or more memory devices after they have been received. In some embodiments the inputs are converted to a standard format prior to storing in the memory device. Operations 122 and 124 can be performed in any order, or simultaneously.

Once the inputs have been received, operation 126 performs the computation of a standard score using the first and second characteristics of the subject that were input. In some embodiments, the computation utilizes a predetermined mathematical formula. The mathematical formula is determined, for example, by using logistic regression to fit a set of data to a logit function logistic curve. The set of data is, for example, a compilation of subject data identifying values of first and second characteristics of patients. In one example embodiment, the data is normalized using log(PSA) and log(PSP94) before analysis.

In one example embodiment, operation 126 computes the standard score (y) using the following formula:

y=A log(PSA)+B log(PSP94)+C.

A, B, and C, are constants. In one example, A is 2.2724, B is −1.4732, and C is 0.3839. Constants can be rounded to fewer significant digits, or expanded to greater significant digits, in some embodiments. Other embodiments use other formulas or other constants, such as those computed from different sets of data associated with different subjects.

In general, for an equivalent number of patients categorized (i.e., for a data set of the same size), one would expect a database divided into three classes (healthy, having non-malignant disease of the prostate, having prostate cancer) to have a greater diagnostic accuracy when used for diagnosing patients, as compared to a database divided into six classes (healthy, having non-malignant disease of the prostate, having localized cancer of the prostate, having metastasised cancer of the prostate, having precancerous prostatic lesions, and having acute or chronic inflammation of prostatic tissue). One would also reasonably expect that an increase in the data characterized (i.e., number of patients entered into the database) would result in an improvement in the diagnostic accuracy of the database. Some embodiments are used for the differential diagnosis of any two or more of the six classes described herein.

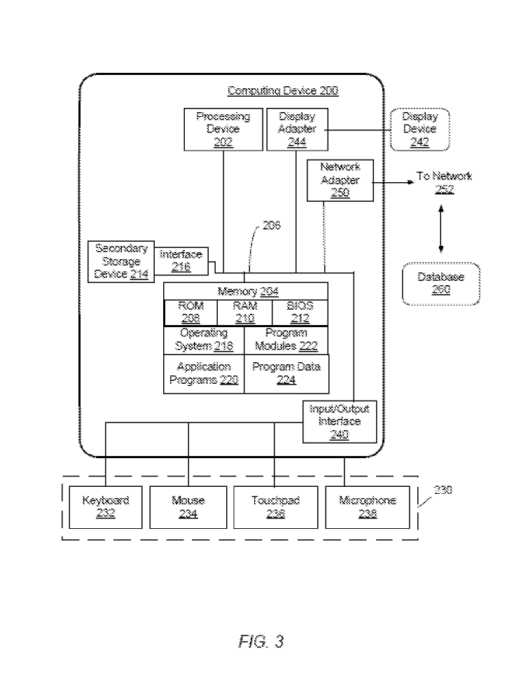

FIG. 3 is a schematic block diagram illustrating an architecture of an example computing device 200 for implementing various aspects according to the present disclosure. The computing device 200 can be used to perform some or all of one or more of the methods, operations, computations, or processes discussed herein, such as those illustrated and described herein with reference to FIGS. 1-2. In addition, some embodiments include two or more computing devices that operate together to perform aspects disclosed herein.

In one example, computing device 200 is a personal computer. Other embodiments include other computing devices 200, such as a tablet computer, a smart phone, a personal digital assistant (PDA), or other device configured to process data instructions. In some embodiments, computing device 200 is an example of programmable electronics. In another possible embodiment, two or more computing devices 200 collectively form at least a portion of the programmable electronics.

Computing device 200 includes, in some embodiments, at least one processing device 202 and memory 204. A variety of processing devices 202 are available from a variety of manufacturers, for example, Intel or Advanced Micro Devices. In some embodiments, the processing device 202 is configured to perform one or more methods or operations as defined by instructions stored in a memory device. Examples of such methods and operations are described herein.

Computing device 104 also includes, in some embodiments, at least one memory device 204. Examples of memory devices 204 include read-only memory 208 and random access memory 210. Basic input/output system 212, containing the basic routines that act to transfer information within computing device 200, such as during start up, is typically stored in read-only memory 208. Memory device 204 can be a part of processing device 202 or can be separate from processing device 202.

In this example, computing device 200 also includes system bus 206 that couples various system components including memory 204 to processing device 202. System bus 206 is one of any number of types of bus structures including a memory bus, or memory controller; a peripheral bus; and a local bus using any of a variety of bus architectures.

In some embodiments, computing device 200 also includes secondary storage device 214 for storing digital data. An example of a secondary storage device is a hard disk drive. Secondary storage device 214 is connected to system bus 206 by secondary storage interface 216. Secondary storage devices 214 and their associated computer readable media provide nonvolatile storage of computer readable instructions (including application programs and program modules), data structures, and other data for computing device 200.

Although the exemplary architecture described herein employs a hard disk drive as a secondary storage device, other types of computer readable media are included in other embodiments. Examples of these other types of computer readable media include magnetic cassettes, flash memory cards, digital video disks, Bernoulli cartridges, compact disc read only memories, digital versatile disk read only memories, random access memories, read only memories, or other memory devices.

A number of program modules can be stored in secondary storage device 214 or memory 204, including operating system 218, one or more application programs 220, other program modules 222, and program data 224. In some embodiments, program modules include data instructions that are stored in computer readable media (such as computer readable storage media). The data instructions, when executed by the processing device 202, cause the processing device 202 to perform one or more of the methods or operations described herein.

In some embodiments, a user provides inputs to the computing device 200 through one or more input devices 230. Examples of input devices 230 include keyboard 232, mouse 234, touchpad 236 (or a touch sensitive display), and microphone 238. Other embodiments include other input devices 230. Input devices 230 are often connected to the processing device 202 through input/output interface 240 that is coupled to system bus 206. These input devices 230 can be connected by any number of input/output interfaces, such as a parallel port, serial port, game port, or a universal serial bus. Wireless communication between input devices and interface 240 is possible as well, and includes infrared, BLUETOOTH® wireless technology, 802.11a/b/g/n/z wireless communication, cellular communication, or other radio frequency communication systems in some possible embodiments.

In some embodiments, a display device 242, such as a monitor, liquid crystal display device, projector, or touch screen display device, is also connected to system bus 206 via an interface, such as display adapter 244. In addition to display device 242, the computing device 200 can include various other peripheral devices (not shown), such as speakers or a printer. When used in a local area networking environment or a wide area networking environment (such as the Internet), computing device 200 is typically connected to network 252 through a network interface or adapter 250. Other possible embodiments use other communication devices. For example, some embodiments of computing device 200 include a modem for communicating across network 252.

Computing device 200 typically includes at least some form of computer-readable media. Computer readable media include any available media that can be accessed by computing device 200. By way of example, computer-readable media include computer readable storage media and communication media.

The term computer readable media as used herein includes computer storage media. Computer storage media includes volatile and nonvolatile, removable and non-removable media implemented in any method or technology for storage of information, such as computer readable instructions, data structures, program modules, or other data. Computer readable storage media includes, but is not limited to, read-only memory 208, random access memory 210, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by computing device 200. In some embodiments, computer readable storage media is non-transitory media.

Communication media can be embodied by computer readable instructions, data structures, program modules or other data in a modulated data signal, such as a carrier wave or other transport mechanism and includes any information delivery media. The term “modulated data signal” refers to a signal that has one or more of its characteristics set or changed in such a manner as to encode information in the signal. By way of example, communication media includes wired media such as a wired network or direct-wired connection, and wireless media such as acoustic, radio frequency, infrared, and other wireless media. In some embodiments, communication media is transitory media. Combinations of any of the above are also included within the scope of computer readable media.

A database 260 is also illustrated in FIG. 3. In some embodiments, database is a separate device from computing device 200, and is in data communication with computing device 200, such as across network 252. In another possible embodiment, however, database 260 is a memory device that is part of computing device 200. In some embodiments, database 260 includes a medical records database. In another embodiment, database 260 includes subject data, such as the compilation of data for a plurality of subjects. Such data can include, for example, one or more of the following for each subject: PSA values, PSP94 values, age, blood pressure values, and Gleason Score values.

The present invention is further illustrated by the following examples, which should not be construed as limiting in any way.

### EXAMPLES

### Example 1

### Samples Used for Biomarker Discovery

Patients were recruited through a series of urological clinics and hospitals located in southern British Columbia (2 sites), Quebec (1 site), Manitoba (1 site), Nova Scotia (1 site) and Ontario (15 sites) for a pre-biopsy screening evaluation. Spot urine samples were collected without a preceding DRE. 24-hour urine samples were obtained prospectively no more than ten days prior to the patient undergoing a previously scheduled biopsy of the prostate for suspicion of prostate cancer. Serum samples were obtained by standard blood draw and collected as a 10 mL sample volume.

Patients were recruited for sample collection for the Pre-Biopsy Screening provided they were able to meet the following criteria:
Patient was male, at least 50 years of age and able to understand, and is willing to sign a written informed consent document.
Patient was previously scheduled for a biopsy of the prostate for suspicion of prostate cancer.
Patient could provide urine samples for analysis and serum samples for total PSA testing.
Patient had complete medical history information available (including tumour stage and grade if the patient was subsequently diagnosed as having prostate cancer).
   Patients were excluded when:
Patient reported a previous incidence of prostate cancer.
Patient reported a previous incidence of non-prostate cancer except basal skin cell carcinoma in the previous two years.
Patient reported taking either investigational agents or any prescribed pre-operative medications at the time of sample collection.

### Patient Clinical/Medical History Information

Medical history information was obtained as close to the time of sample collection as possible. This information included: age of patient; circulating PSA levels at time of sample collection; pathology and history of prostate cancer; presence of other chronic or acute conditions unrelated to prostate cancer at the time of sample collection and current management as well as current and past treatment regimes for prostate cancer.

### Sample Groups

Aggressive prostate cancer was defined as Gleason score of ≧7 and non-cancer/non-aggressive cancer was defined as Gleason score ≦6. Non-cancer samples included:
Patients diagnosed with a non-malignant disease of the prostate (for example, benign prostatic hyperplasia): confirmation of the absence of prostate cancer was evaluated by histological examination of prostatic tissue (needle point biopsy).
Prostatic intraepithelial neoplasia (PIN) samples: patients were diagnosed as having the disease by confirmation of the presence of PIN through post-surgical histological evaluation (biopsy).
Non-PCa/PIN samples: patients were diagnosed as being free of disease by confirmation of the absence of prostate cancer/PIN as evaluated by histological examination of prostatic tissue (biopsy).
Control samples: patients with no reported complaints or symptoms related to prostate cancer, and who were not suffering from severe disease at the time of collection.

TABLE 1
Patient Samples test for PSP94 Total PSA values from
2.5 to 10 ng/mL

| | | Non- | | |
|---|---|---|---|---|
| | | Cancer/Non- | | |
| | | Aggressive | | |
| | Aggressive | Cancer | | |
| | Prostate | Gleason | Non | |
| | Cancer | score 6 | Cancer | Total |
| Total | 18 | 26 | 44 | 88 |
| Samples in | | | | |
| Study | | | | |
| Men without | 7 | 15 | 22 | 44 |
| hypertension | | | | |

### Sample Handling

Samples originating from sites outside of Winnipeg, Manitoba were shipped frozen on dry ice. Those samples obtained from Victoria General Hospital (Winnipeg, Manitoba) were frozen at the site and then transported on dry ice to the laboratory. Those samples obtained from the Winnipeg Clinic were stored at 4° C. at the site for same-day pickup. These samples were then transported on ice to the laboratory. Upon receipt urine samples stored at −20° C.

### Example 2

### Immunoassay of Urine PSP94 in a Microsphere Multiplex System

Of 4 peaks detected by mass spectrometry in the biomarker discovery set of samples, only 1 was statistically significant both in the original set of samples and the validation set for both Control vs PCa and BPH vs PCa. Prostate specific protein 94 (PSP94) with a predicted mass of ˜10,770 Da was identified by LC-MSMS as the one biomarker of 10,750 Da in the validation samples and it co-eluted during purification with the biomarker of ˜10,657 Da.

After identifying PSP94 as the biomarker of interest form mass spectrometry, we created a sandwich immunoassay to detect PSP94 on a BioPlex® platform and characterized the analytic performance of the test.

### Microsphere Preparation:

Microspheres were coated with 10 μg polyclonal anti-PSP94 antibody (R&D Systems, Minneapolis, Minn.) per 1,250,000 beads using the BioRad® coupling procedure for the Amine Coupling Kit.

### Preparation of Calibrators and Controls:

PSP94 obtained from R&D Systems was diluted in Assay Buffer to create a 1000 ng/mL stock solution. Dilutions were made from the 1000 ng/mL stock to create calibrators and controls that ranged from 0.5 ng/mL to 12 ng/mL.

Sample Preparation before Testing:

Aliquoted urine samples were stored at −20° C. On the day of immunoassay testing, the thawed samples were centrifuged for 4 minutes at 13,000 RPM (16,000×g) on a Heraeus® Biofuge. The samples were diluted to 1:20 in a PBS Buffer containing bovine serum albumin (assay buffer).

### Immunoassay to Detect of PSP94 in Urine:

This assay used a quantitative sandwich enzyme immunoassay format.
1. The polyclonal antibody specific for PSP94 was coupled onto microspheres, and the microspheres were vortexed and sonicated. Fifty microliters (50 μL) of resuspended microspheres were then pipetted into a Millipore® microfilter plate and washed with PBS and 0.05% Tween® buffer (Wash Buffer).
2. The washed beads were followed by the addition of 50 μL of either PSP94 calibrators (0 to 12 ng/mL), controls (2 or 6 ng/ml) or urine samples diluted into Assay Buffer. PSP94 present in urine bound to the polyclonal antibody attached to the microspheres.
3. After washing away unbound substances with Wash Buffer, a 1:400 dilution of a mouse anti-PSP94 monoclonal antibody (Novus Biologicals) in Assay Buffer was added to each well.
4. After washing away unbound substances with Wash Buffer, a 1:100 dilution of a goat anti-mouse antibody linked to phycoerythrin (Jackson Immunoresearch) in Assay Buffer was added to each well. The wells were washed with Wash Buffer and resuspended in 130 uL of Assay Buffer.
5. The filter plates were placed in the BioPlex® 200 to quantify the fluorescence from the goat anti-mouse PE bound to beads. The fluorescence intensity was proportional to the concentration of PSP94 in urine.
6. Quantitation of results were determine by a 4-PLC curve fit from

### Example 3

### Analytical Performance

### Typical Calibration Curve:

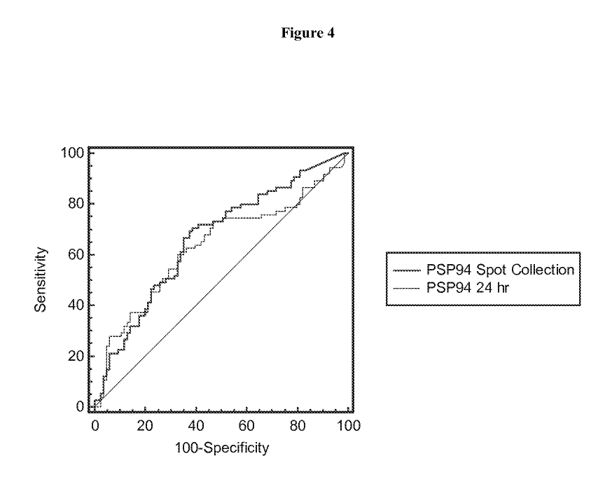

The average of duplicate readings for each calibrator control and sample were calculated using the BioPlex® 200 software to generate a 4-PLC curve fit. The zero calibrator did not need to be subtracted from the other calibrators, controls or samples for accurate results. A typical curve is shown in FIG. 4 and typical results for each calibrator are shown in Table 2.

TABLE 2
Typical Calibration Curve

| Concentration | | | Concentration | |
|---|---|---|---|---|
| (ng/mL) | Fluorescence | % CV | (ng/ml) | (Obs/Exp) * 100 |
| 12 | 2875.3 | 1.17 | 11.22 | 94 |
| 8 | 2535 | 4.18 | 8.6 | 107 |
| 4 | 1432.5 | 3.55 | 3.99 | 100 |
| 1 | 274.3 | 3.74 | 0.97 | 97 |
| 0.5 | 125.5 | 4.51 | 0.52 | 103 |
| 0 | 25.8 | 4.12 | | |

### Intra-Assay Precision:

Controls were run in duplicate or triplicate over 13 different plates for over 1 month. The intra-assay precision for the 2 and 6 ng/mL PSP94 was less than 5% and the inter-assay precision is less than 10%.

### Spike Recovery:

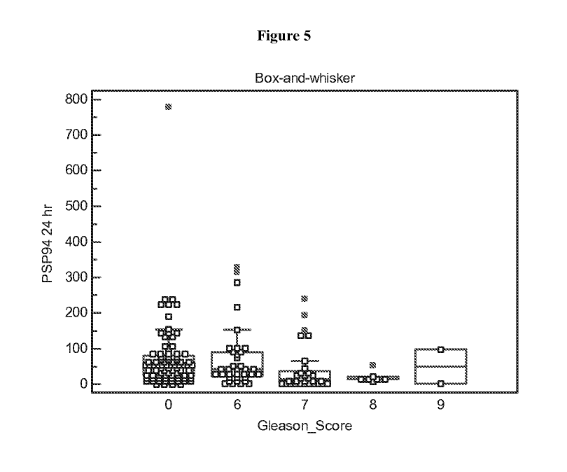

An example of recovery is provided in FIG. 5 where a sample with 1.6 ng/mL PSP94 in 1:10 diluted urine was spiked with 3 and 10 ng/mL PSP94. The % recovery was 95% for spiked samples. The slope is approximately 1.0 with a correlation coefficient of ˜1.0 indicating near perfect recovery of PSP94 in this study.

### Analytical Sensitivity:

Limit of Quantitation (LOQ) was estimated with 5 different standard assays using multiple bead preparations and reagent preparations. Specifically, the LOQ was calculated by determining the concentration of PSP94 from a 4-PLC curve fit for the

Fluorescence=mean of the zero calibrator+10*standard deviation of the zero calibrator.

The LOQ ranged from 0.10 to 0.15 ng/mL. At a 1:20 dilution, the LOQ was 2.0 to 3.0 ng/mL.

### Specificity:

PSA was added at 100 ng/mL to the zero calibrator. The concentration of PSP94 was less than the LOQ. Therefore, PSA did not interfere with this assay.

### Freeze-Thaw Cycles:

There was no effect (total CV from 4-11% CV) of up to 4 freeze thaw cycles on the performance of PSP94 when tested on 5 fresh samples with PSP94 data ranging from 8 to 220 ng/mL.

### Example 4

### PSP94 alone and PSP94 Plus PSA for the Diagnosis of Prostate Cancer

Samples were collected from men according to Example 1. Spot urine samples were tested for PSP94. Matched serum samples were then analyzed for PSA. Data were then analyzed using MedCalc Software version 9.5.2.0 (2008) (MedCalc Software bvba, Belgium). PSP94 on the Bioplex® was diagnostic for Controls vs PCa; (p=0001) and BPH vs PCa (p=0010) using a pre-DRE spot collection. These data confirm that PSP94 was diagnostic for prostate cancer.

We further explored the data by examining Gleason scores combined with PSP94 values obtained from pre-DRE spot collections and 24 hr collections from the same men. We observed that a Gleason Score of 7, 8 and 9 had decreased values of PSP94 compared to a Gleason Score of 6 or noncancer. An example of this is shown in FIG. 5 for PSP94 at 24 hr. When we examined points that were above the whiskers in FIG. 5, we observed that 5 of 6 patients had hypertension. No other disease correlated as well for these samples.

TABLE 3
Six patients that had elevated PSP94 above the whiskers for Gleason score 7 and 8.

| Patient | | | |
|---|---|---|---|
| No | Medical History | Meds | Gleason Score |
| 2016 | Hypertension, high cholesterol, | Detrol LA, Norvasc, Avandia, | 4 + 4 = 8 |
| | diabetes, BPH, glasses | Glyburide, Novo-Hydrazide, | |
| | | Metformin, Vasotec, Novo- | |
| | | Terazosin, Lipitor, Betoptic | |
| 2015 | Hypertension; gastrointestinal | Pariet (gastric reflux; 20 mg OD); | 4 + 3 = 7; 4/10 |
| | (Diverticulitis); has trouble with | HCT2 (hypertension 12.5 mg); | positive cores |
| | organism, Asthma, Decreased flow of | Synthroid (thyroid 0.05 mg); | |
| | urine, Allergies to flagyl, trees, grasses, | Symbicant (asthama); Calcium | |
| | Depression, Benign basil skin | puffer (general bone health); | |
| | carcinoma (7 yrs ago), grandmother and | Cipro (gastrointestinal flare-ups) | |
| | mother had breast cancer, father had | | |
| | prostate cancer, sister had bone cancer | | |
| 2092 | pulmonary embolism-1996, phlebitis- | Coumadin, Nexium, Exetrol, | 3 + 4 = 7 |
| | 1996, hypercholesterolemia, heart burn, | Zocor, Acetaminophene | |
| | Sister skin cancer on scalp | | |
| 2099 | Hypertension, heart burn, protatis, | Aspririn, Lipitor, Norvasc, Periet, | 3 + 4 = 7 |
| | vasectomy, elevated cholesterol, frozen | Selexin, Vitamin B, Vitamin C | |
| | shoulder, itchy head, allergies, | | |
| | hemmoroids, inflammed groin, fell and | | |
| | had a bruised testicle | | |
| 2160 | Hypertension, high cholesterol, | Arthrofec, ASA, Betamethasone, | 3 + 4 = 7 |
| | indigestion, hernia, high iron (in blood), | Coversyl, Elocom cream, Lipitor, | |
| | psorasis, Mother breast cancer, Father | Losec, Metformin, Tylenol 3 | |
| | liver cancer, Brother brain & bowel | | |
| | cancer | | |
| 2163 | Hypertension; alleries to MSG | Pariet, Hydroxyquine, Naproxen, | 4 + 3 = 7 & 4 + 4 = 8 |
| | | Vitamin D, Prednisone | |

Based on these observations, we wanted to determine if hypertension interferes with PSP94 quantitation. To test whether hypertension interferes, we used the area under the curve (AUC) for the ROC curve to examine the diagnostic capability of the test. An AUC of 1.0 is diagnostically perfect, and an AUC of 0.50 is has no diagnostic capability. Therefore, an AUC that is statistically significant above 0.50 indicates an improvement in diagnostic capability of the test with values close to 1.0 having a higher diagnostic significance. When we examined the ability of PSP94 in spot collected urine to diagnose prostate cancer or high Gleason Score (HGS) from non-cancer patients, we observed that men without hypertension had statistically significant AUC above 0.50, which indicated the diagnostic capability of the test. In contrast, men with hypertension did not have statistically significant AUC for non-cancer compared to prostate cancer (0.612) or non-cancer versus HGS (0.626). PSP94 samples from urine collection at 24 hr had similar results as the PSP94 Spot Collection. PSP94 in urine samples at 24 hr from non-hypertensive males were diagnostic for non-cancer versus prostate cancer (0.680) and non-cancer versus HGS (0.847) but were not diagnostic for men with hypertension.

TABLE 4
The area under the curve (AUC) for PSP94 and PSA when examining
the diagnostic effectiveness of non-cancer compared to either prostate
cancer or high Gleason score when men were categorized as having
either normal blood pressure or hypertension.

| | PSP94 Spot | | PSP94 24 hr | | | |
|---|---|---|---|---|---|---|
| | Collection | | Collection | | PSA | |
| | (ng/mL) | | (ng/mL) | | (ng/mL) | |
| | Normal | | Normal | | Normal | |
| | Blood | Hyper- | Blood | Hyper- | Blood | Hyper- |
| | Pressure | tension | Pressure | tension | Pressure | tension |
| noncancer vs | 0.711* | 0.612 | 0.680* | 0.572 | 0.664* | 0.739* |
| PCa | | | | | | |
| Noncancer vs | 0.773* | 0.626 | 0.847* | 0.596 | 0.726* | 0.757* |
| HGS | | | | | | |

*p < 0.05

A possible explanation for the distinction between men with and without hypertension was sampling bias. To eliminate the possibility of sampling bias, we demonstrated that another biomarker in the same men did not demonstrate the observed “hypertension effect.” Therefore, we used a general screening test to measure total PSA measurements as a control to verify that the data set was not biased. Table 4 indicates that PSA is statistically diagnostic in non-cancer versus prostate cancer and non-cancer versus HGS in men with or without hypertension. Thus, the lack of diagnosis for prostate cancer by measuring PSP94 is not due to a bias in the data and strongly indicates that hypertension affects PSP94 measurements.

Next we next explored the ability of PSP94, PSA, age, smoking history and BMI to be combined for potential screening tests. There was also no significant correlation between PSP94, PSA, age, smoking history and BMI with the exception of a weak (r=−0.205) correlation between BMI and age. Thus, it was possible to combine these diagnostic and clinical characteristics with logistic regression to improve diagnostic capabilities. For logistic regression, we used log(PSA) and log(PSP94) to normalize the data first before analysis. We examined HGS and PCa diagnoses with the algorithms created by logistic regression. Improvements in the AUC for the ROC curves were observed (Table 5) when compared to either PSA or PSP94 results alone.

TABLE 5
AUC for the ROC curves from logistic regression models for the
diagnosis of non-cancer from either PCa or HGS using PSA and PSP94
in men with normal blood pressure.

| | PSP94 Spot and PSA | PSP94 24 hr |
|---|---|---|
| | Collection (ng/mL) | Collection (ng/mL) |
| | Normal Blood | Normal Blood |
| | Pressure | Pressure |
| noncancer vs PCa | 0.773 | 0.714 |
| Noncancer vs High | 0.881 | 0.881 |
| Gleason Score* | | |

All models AUC had p < 0.0001 compared to AUC = 0.50.

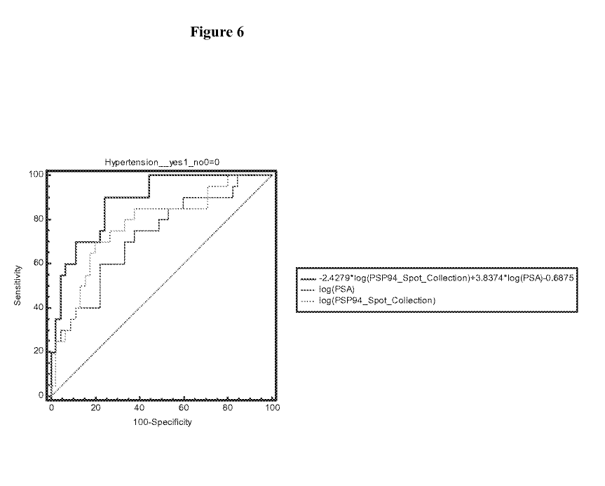

For example, the AUC of the ROC curve for the logistic regression of PSP94 spot collection and PSA for HGS versus noncancer samples from men with normal blood pressure were statistically higher compared to either PSP94 spot collection (p=0.048) or PSA (p=0.013) alone (FIG. 6). These data indicate that PSP94 and PSA results can be combined to improve the diagnosis HGS compared PSA alone. This improvement is demonstrated in Table 6 where a ˜2.5 fold improvement in prostate cancer detection with logistic regression model of PSP94 and PSA compared to PSA alone.

TABLE 6
Detection in Prostate Cancer at Specificity of ~77% by PSA or
algorithm of PSP94 spot collection and PSA.

| | | | Gleason | |
|---|---|---|---|---|
| | | Non Cancer | Score 6 | HGS |
| | Cutoff | TN/(TN + FN) | TP/(TP + FP) | TP/(TP + FP) |
| PSA | >10 ng/mL | 35/45 (78%) | 4/22 (18%) | 9/20 (45%) |
| Algorithm | ≧−1.2848 | 34/45 (76%) | 12/22 (55%) | 18/20 (90%) |

TP = true positive;
FP = false positive;
TN = true negative;
FN = false negative

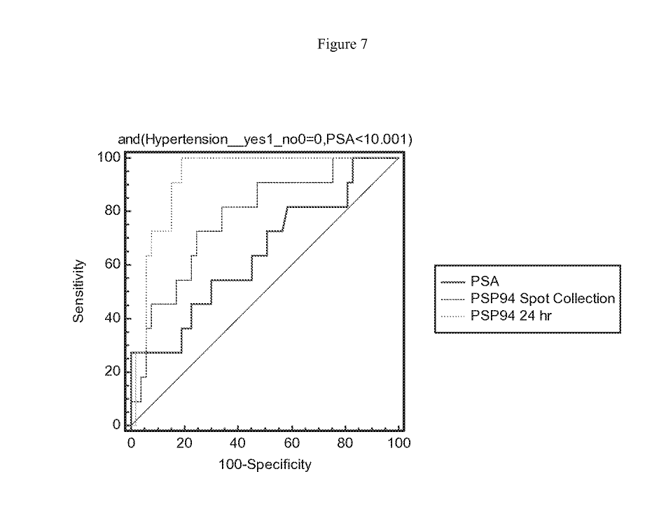

From Table 6, we were able to provide two flow charts of expected results from 100 men going to a urology clinic in Canada. FIG. 7 demonstrates the current PSA test with a high level of specificity which exists at >10 ng/mL that would decrease the number of unnecessary biopsies. Under these conditions, PSA would miss 33 of 48 men with cancer. PSA normally is selected at 4 ng/mL to detect men with cancer, however, it comes at the expense of a >70% of unnecessary biopsies. However, when we combined PSA with PSP94 spot collection, we achieved both high sensitivity and specificity where we detected 90% of all cancers in high Gleason score and the majority of Gleason score 6 samples. The diagnostic capability of the algorithm is the total correct diagnoses compared to the total samples tested. The diagnostic capability of the algorithm is 74% compared to PSA at 55%.

We also examined the diagnosis of PCa and HGS with PSP94 in men with ≦10 ng/mL PSA. This cutoff was selected since men with >10 ng/mL PSA are normally selected for biopsy. The hypothesis is that PSP94 would improve the diagnosis of high Gleason score prostate cancer compared to men with 0 to 10 ng/mL PSA and the results are shown in Table 7. Men with ≦10 ng/mL PSA would benefit from improvements with a PSP94 test where PSP94 in spot collection (p=0.017) and PSP94 in 24 hr urine (p=0.009) had statistically significant improvement in the AUC compared to PSA. PSP94 spot collection and PSP94 in 24 hr urine where the PSP94 in 24 hr urine is close to an AUC of 1.0 and has a 100% sensitivity and 78% specificity. When 100 men with normal blood pressure and with PSA <10 ng/mL go to a Canadian clinic, 54 men would have unnecessary biopsies if being tested for high Gleason Score prostate cancer and 4 men would not be detected for HGS with a PSA cutoff value of >4.0 ng/mL. This is in contrast to PSP94 in 24 hr urine where only 15 unnecessary biopsies and detected all HGS when PSP94 in 24 hr urine was <28.7 ng/mL. When we examined the capability of separating HGS prostate cancer from all other samples in men with PSA values below 10.00 ng/mL and normal blood pressure, we observed a 85% diagnostic capability for PSP94 in 24 hr urine collection while PSA from 0 to 10.0 ng/mL had a 42% diagnostic capability and the number of unnecessary biopsies would decreases from 54 with PSA to 15 with PSP94.

TABLE 7
AUC for the ROC curves from data with PSA values from
0-10.0 ng/mL from men with normal blood pressure.

| | PSP94 Spot | 24 hr Collection | PSA |
|---|---|---|---|
| | Collection (ng/mL) | (ng/mL) | (ng/mL) |
| noncancer vs PCa | 0.694 | 0.680 | 0.708 |
| Noncancer vs High | 0.833 | 0.914 | 0.723 |
| Gleason Score | | | |

In these studies, we observed that both PSP94 values from spot collection and PSP94 24 hour collection can be combined with results from PSA to improve screening for men with high Gleason score (≧7) compared to non-cancer. This can occur by creation of algorithms of PSA with PSP94 in spot collection or by the selecting patients with PSA values from 0 to 10 ng/mL. The combination of PSP94 with PSA can identify high Gleason score patients most at risk for aggressive prostate cancer and also reduce the number of unnecessary biopsies in men. PSP94 test in combination with PSA would also be useful in active surveillance where men have low Gleason score prostate cancer but would like an non invasive test to determine when they are at risk for more aggressive prostate cancer at higher Gleason scores (>=7) and thus minimize the number of biopsies required in the future.

We claim:

1. A method of diagnosing prostate cancer in a subject, comprising:
(a) detecting a quantity, presence, or absence of PSP94 in a first biological sample from the subject;
(b) optionally detecting total PSA in the first biological sample or a second biological sample from the subject; and
(c) comparing the quantity, presence or absence of PSP94 and optionally total PSA as detected in steps (a) and (b) with a standard score.

2. The method of diagnosing prostate cancer in a subject of claim 1, wherein said standard score is obtained from one or more subjects known to have prostate cancer, wherein a similarity in quantity, presence, or absence of PSP94 and optionally total PSA between the quantity, presence or absence as detected in steps (a) and (b) with said standard score results in a diagnosis of prostate cancer in the subject.

3. The method of diagnosing prostate cancer in a subject of claim 1, wherein said standard score is obtained from one or more known subjects having a Gleason score of less than or equal to 6, wherein a deviation in quantity, presence, or absence of PSP94 and optionally total PSA between the quantity, presence or absence as detected in steps (b) and (c) with said standard score results in a diagnosis of aggressive prostate cancer in the subject.

4. The method of diagnosing prostate cancer in a subject of claim 1, wherein said standard score is obtained from one or more subjects known to have a Gleason score of greater than or equal to 7, wherein a similarity in quantity, presence, or absence of PSP94 and optionally total PSA between the quantity, presence or absence as detected in steps (b) and (c) with said standard score results in a diagnosis of aggressive prostate cancer in the subject.

5. The method of diagnosing prostate cancer in a subject of claim 1, wherein said standard score is obtained from one or more subjects known to be selected from the group consisting of (i) healthy subjects, (ii) subjects having a precancerous prostatic lesion, (iii) subjects with non-malignant disease of the prostate, (iv) subjects with localized cancer of the prostate, (v) subjects having an acute or chronic inflammation of prostatic tissue (v) subjects with metastasised cancer of the prostate, wherein a similarity or difference between the quantity, presence or absence of PSP94 and optionally the total PSA in the first or the first and second biological samples and the standard score is used to determine whether the subject is healthy or has a precancerous prostatic lesion, a non-malignant disease of the prostate, a localized cancer of the prostate, an acute or chronic inflammation of prostatic tissue, or a metastasised cancer of the prostate.

6. The method of diagnosing prostate cancer in a subject of claim 1, wherein said standard score is obtained from the subject in the past, wherein a deviation in quantity, presence, or absence of PSP94 and optionally total PSA between the quantity, presence or absence as detected in steps (a) and (b) with said standard score results in a indicator of the progression of the prostate cancer in the subject.

7. The method of any one of claims 1-6 further comprising the step of diagnosing whether the subject has hypertension.

8. The method of any one of claims 1-6 wherein the subject does not have hypertension.

9. The method of any one of claims 1-8 wherein the detection of the quantity, presence or absence of PSP94 and optionally total PSA comprises the steps of:
(a) contacting the biological sample with a biologically active surface; and
(b) allowing the PSP94 and optionally PSA within the biological sample to bind to the biologically active surface.

10. The method of any one of claims 1-8 wherein the detection of the quantity, presence or absence of PSP94 and optionally total PSA comprises the steps of:
(a) contacting the biological sample with one or more binding molecule specific for PSP94 and PSA; and
(b) detecting the quantity, presence or absence of PSP94 and optionally PSA,

11. The method of any one of claims 1-10 wherein the subject is diagnosed as having aggressive prostate cancer when the quantity of PSA is determined to be between 0.0-10 ng/ml.

12. The method of any one of claims 1-11 wherein the quantity, presence, or absence of PSP94 and total PSA are detected by utilizing an antibody specific to PSP94 or total PSA.

13. The method of any one of claims 1-12 wherein the quantity, presence, or absence of PSP94 and total PSA are detected by utilizing an ELISA assay.

14. The method of any one of claims 1-13 wherein the quantity, presence, or absence of PSP94 and total PSA are detected through use of a multiplex immunoassay.

15. The method of any one of claims 1-14 wherein the biological sample is selected from the group consisting of whole blood, blood serum, blood plasma, urine, semen, seminal fluid, seminal plasma, prostatic fluid, pre-ejaculatory fluid (Cowper's fluid), excreta, tears, saliva, sweat, biopsy, ascites, cerebrospinal fluid, lymph, and a biopsy sample.

16. The method of claim 15 wherein the biological sample is urine.

17. The method of any one of claims 1-16 wherein the sample is collected by spot collection.

18. The method of any one of claims 1-17 wherein the sample is collected by 24 hour collection.

19. A kit for diagnosing prostate disease in a subject comprising: a biologically active surface comprising an adsorbent, binding solutions, and instructions to use the kit; wherein the instructions outline a method for diagnosis of a prostate cancer in a subject according to the invention or a method for the differential diagnosis of healthy, non-malignant disease of the prostate, precancerous prostatic lesion, localized cancer of the prostate, metastasised cancer of the prostate, and acute or chronic inflammation of prostatic tissue in a subject according to the method of any one of claims 1-19.

20. The kit of claim 19 comprising a biologically active surface comprising an adsorbent comprised of silicon dioxide molecules.

21. The kit of claim 20 comprising a biologically active surface comprising an adsorbent comprising antibodies specific to PSP94 and optionally PSA.

22. A method of computing a standard score, the method comprising:
receiving with programmable electronics a first input identifying a first characteristic of a subject, the first input including a PSA value;
receiving with the programmable electronics a second input identifying a second characteristic of the subject, the second input including a PSP94 value; and
computing with the programmable electronics a standard score using the first and second characteristics of the subject and storing the standard score in a memory device.

23. The method of claim 22, further comprising determining if the subject has hypertension, and computing the standard score only if the subject does not have hypertension.

24. The method of claim 22, further comprising:
receiving a blood pressure input; and
determining whether the subject has hypertension using the blood pressure input; and;
computing the standard score only if determined that the subject does not have hypertension.

25. The method of claim 24, further comprising prompting the user for the first and second inputs only if determined that the subject does not have hypertension.

26. The method of claim 22, wherein the programmable electronics include at least one processor and the least one memory device.

27. The method of claim 26, wherein the programmable electronics include at least two processors.

28. The method of claim 27, wherein the at least two processors are in data communication across a data communication network.

29. The method of claim 22, wherein receiving the first and second inputs comprises receiving the first and second inputs with an input device of a computing device.

30. The method of claim 22, further comprising displaying the standard score on a display device of the programmable electronics, wherein the programmable electronics include a computing device.

31. The method of claim 22, further comprising saving the standard score in a medical record of a subject in a medical records database.

32. The method of claim 22, further comprising sending the standard score to a computing device across a network.

33. The method of claim 22, wherein computing a standard score comprises calculating the results of a mathematical formula.

34. The method of claim 33, wherein the mathematical formula is based on fitting a set of data to a logit function logistic curve using logistic regression.

35. The method of claim 33, wherein the set of data includes data for a plurality of subjects, the data including at least a PSA value and a PSP94 value for each subject.

36. The method of claim 22, wherein computing a standard score comprises computing a result of:
y=A log(PSA)+B log(PSP94)+C
where y is the standard score; PSA is the PSA value; PSP94 is the PSP value; and A, B, and C are constants.

37. The method of claim 36, wherein A is 2.2724, B is −1.4732, and C is 0.3839.

38. The method of claim 22, further comprising determining if the subject has hypertension, and computing the standard score only if the subject does not have hypertension.

39. The method of claim 22, wherein the standard score is adapted to be used for subject diagnosis.

40. The method of claim 22, wherein the standard score is adapted to be used for prostate cancer diagnosis.

41. A computer-readable storage medium comprising instructions that, when executed by a computer, cause the computer to:
receive a first input identifying a first characteristic of a non-hypertensive subject, the first input including a PSA value;
receive a second input identifying a second characteristic of the subject, the second input including a PSP94 value; and
compute with a computing device a standard score using the first and second characteristics of the subject and storing the standard score in a memory device.

42. The computer-readable storage medium of claim 22, wherein the first input is received with the computing device with an input device.

43. The computer-readable storage medium of claim 22, wherein the first input is received from a second computing device.

44. A method of diagnosing a subject, the method comprising:
determining if the subject has hypertension;
if the subject does not have hypertension, receiving a biological sample from the subject;
determining a first characteristic of the subject from the biological sample, the first characteristic including a PSA value;
determining a second characteristic of the subject from the biological sample, the second characteristic including a PSP94 value;
computing a standard score based at least in part on the PSA value and the PSP94 value; and
diagnosing prostate cancer in the subject using the standard score.

45. The method of claim 44, wherein computing the standard score comprises computing a result of:
y=A log(PSA)+B log(PSP94)+C
where y is the standard score; PSA is the PSA value; PSP94 is the PSP94 value; and A, B, and C are constants.

46. The method of claim 45, wherein A is 2.2724, B is −1.4732, and C is 0.3839.

47. A system comprising:
at least one processor; and
memory, the memory storing instructions that, when executed by the processor, cause the processor to:
receive a first input identifying a first characteristic of a non-hypertensive subject, the first input including a PSA value;
receive a second input identifying a second characteristic of the subject, the second input including a PSP94 value; and
compute with a computing device a standard score using the first and second characteristics of the subject and storing the standard score in a memory device.
